(19) United States
(12) Patent Application Publication
(10) Pub. No.: US 2022/0348624 A1
(43) Pub. Date: Nov. 3, 2022

(54) RECOMBINANT INTERLEUKIN-15 ANALOG

(71) Applicant: LETO LABORATORIES CO., LTD, Beijing (CN)

(72) Inventors: Yao ZHAO, Beijing (CN); Jianjun ZHANG, Beijing (CN); Xiaoting ZHU, Beijing (CN); Yanling XU, Beijing (CN); Lujia PENG, Beijing (CN); Jishu WANG, Beijing (CN); Wei ZHANG, Beijing (CN)

(73) Assignee: LETO LABORATORIES CO., LTD, Beijing (CN)

(21) Appl. No.: 17/763,227

(22) PCT Filed: Sep. 24, 2020
(86) PCT No.: PCT/CN2020/117279

§ 371 Date: Mar. 24, 2022

(30) Foreign Application Priority Data
Sep. 25, 2019 (CN) 2019 10910158.5

(51) Int. Cl. C07K 14/54; C12N 15/70

(52) U.S. Cl. CPC C07K 14/5443; C12N 15/70

(57) ABSTRACT

The amino acid sequence of the present IL-15 analog includes the amino acid sequence of IL-15 and an amino acid sequence including at least one positively charged amino acid added to the C-terminal of the amino acid sequence of IL-15. The present IL-15 analog is highly expressed in Escherichia Coli, wherein the expression level is about 20-fold higher than that of the wild-type IL-15, and there is no significant difference in cell activity in vitro. In addition, a conjugate of the IL-15 analog improves the half-life and the long-term efficacy of the IL-15 analog by coupling with the fatty acid chain. These improvements lay a foundation for the industrialization of IL-15 protein drugs.

### CROSS REFERENCE TO THE RELATED APPLICATIONS

This application is the national phase entry of International Application No. PCT/CN2020/117279, filed on Sep. 24, 2020, which is based upon and claims priority to Chinese Patent Application No. 201910910158.5, filed on Sep. 25, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBSHJL008_Sequence Listing.txt, created on 03/14/2022 and is 107,999 bytes in size.

### TECHNICAL FIELD

The present invention belongs to the field of molecular biology, and particularly relates to a recombinant interleukin-15 analog and expression thereof.

### BACKGROUND

Interleukin-15 (IL-15) is a cytokine having a size of about 12 to 14 kD. The mature peptide of natural human interleukin-15 contains 114 amino acids, including 4 cysteine residues. Two pairs of intramolecular disulfide bonds respectively formed by the connection of Cys35 and Cys85 and the connection of Cys42 and Cys88 play an important role in maintaining the spatial conformation and biological activity of IL-15.

Similar to most cytokines, IL-15 plays a role in normal immune responses, such as promoting the development of T cells, B cells and natural killer (NK) cells. IL-15 and IL-2 share the same β chain and γ chain receptors, thus their biological activities are very similar. Due to the different a chain receptors, IL-2 can activate Treg cells and induce Teff and NK cell apoptosis (AICD), thus the clinical application of IL-2 is greatly limited. In contrast, IL-15 of the same family does not have the functions of Treg activation and AICD, thus IL-15 has great therapeutic potential.

The a subunit of the IL-15 receptor has a high affinity for IL-15. Under physiological conditions, IL-15 mostly forms a complex (IL-15-Ra) with the a subunit, which enhances the affinity of IL-15 for the β chain subunit and the γ chain subunit of the receptor, and activates T cells and NK cells. Thereby, companies such as ALTOR and Hengrui have utilized the characteristics of the a subunit to form a complex with IL-15 and a subunit (or its part) through fusion or non-fusion methods, which has shown good biological potency and stability in animal experiments.

The α subunit of IL-15 receptor is expressed on the surface of myeloid cells, including macrophages, antigen-presenting cells, NK cells and T cells. It also plays an important role in anti-tumor in vivo. Therefore, the IL-15-Ra complex is independent of the α subunit in vivo. Although the stability of IL-15 monomer is improved, it has a subtle functional difference from native IL-15, which may reveal essential differences in antitumor in vivo.

IL-15 has higher safety and activity as compared to IL-2. However, as a protein drug, natural wild-type IL-15 has significant drug development bottlenecks, including low expression levels in prokaryotes and eukaryotes, difficult purification, and short half-life, making it difficult to be industrialized. Therefore, it is necessary to study the exogenous expression of IL-15 to obtain high production efficiency. In addition, the N-terminal or C-terminal of IL-15 analog obtained by in vitro renaturation can be coupled to the fatty acid chain of human serum albumin binder, so as to achieve a long-term efficacy, which has certain significance for the treatment of tumors.

### SUMMARY

In view of the low production efficiency of IL-15 in the prior art, the present disclosure provides an IL-15 analog with high expression level and relatively simple purification process. Further, the present disclosure provides a conjugate of the IL-15 analog, thus as to improve the half-life of IL-15 and the long-term efficacy thereof.

In the first aspect, the present disclosure provides an IL-15 analog. In a particular embodiment, the amino acid sequence of the IL-15 analog comprises the amino acid sequence of IL-15, and one or more amino acids added to the C-terminal of the amino acid sequence of IL-15.

Preferably, the aforementioned one or more amino acids comprise positively charged amino acid.

Preferably, the amino acid sequence of the IL-15 analog is characterized by:

IL-15-Xₐ-Y_{b}-Z_{c}

wherein X, Y and Z each represent an amino acid sequence added at the C-terminal, and a, b and c each represent the number of the amino acids; and

wherein X and Z each are any amino acid or a combination of any amino acids, and a and c each are 0 to 20; and wherein Y is a positively charged amino acid or a combination of any positively charged amino acids, or a combination of a positively charged amino acid and any other amino acids, and b is 1 to 7.

Preferably, the positively charged amino acid is H, R or K.

Optionally, the aforementioned X comprises any one of V, I, P, L, E, A, S, C, T and G, or a combination thereof.

Optionally, the amino acid sequence of the IL-15 analog is characterized by:

IL-15-linker-Xₐ-Y_{b}-Z_{c}

wherein the linker represents a linker sequence between the amino acid sequence of IL-15 and the amino acid sequences added to the C-terminal thereof.

Preferably, the linker is (GGGGS)ₙ (SEQ ID NO: 120), (GS)ₙ or (GAPQ)ₙ (SEQ ID NO: 121), with n being 0 to 10.

Preferably, Xₐ comprises LPBTG (SEQ ID NO: 151) with B being any amino acid, and the linker is (GS)ₙ.

Optionally, the amino acid sequence added to the C-terminus of the IL-15 analog is selected from the group shown in Table 1.

TABLE 1
The structure of the amino acid sequence added to
the C-terminus of the IL-15 analog

| Amino Acid Sequence | SEQ ID NO: |
|---|---|
| GSGSGS-HHHHHH | 122 |
| GS-HHHHHH | 123 |
| PLASTKKR | 124 |
| LPKSAKKK | 125 |
| KKKKKKK | 126 |
| (GAPQGAPQ)-LVESAHHH | 127 |
| GS-LVSSAHHK | 128 |
| GS-LIEHHRRK | 129 |
| GS-IVEHRKKK | 130 |
| GS-VPKTGRRR | 131 |
| GS-LVASGKK | 132 |
| GS-HRKSGHHH | 133 |
| GS-LPKTGRHK | 134 |
| KKKTGRRH | 135 |
| LPRSGRHK | 136 |
| LVETHHHH | 137 |
| VRPETHHH | 138 |
| KKK | |
| RHHHH | 139 |
| KRETHHHH | 140 |
| GS-LPETG-GSGGSHHHHHH | 141 |
| HLETGKKK | 142 |
| HVESGRRR | 143 |
| RRHTGKKK | 144 |
| HVKTGHHH | 145 |
| HVKTGHHH | 146 |
| HVKSSHRH | 147 |
| GSGSGSGSGS-LVKSGHHH | 148 |
| RPKSGHHK | 149 |
| KKC | |
| LHKAGKHH | 150 |
| K | |
| KK | |

Preferably, the amino acid sequence of IL-15 is selected from the group of:

1) the amino acid sequence shown in SEQ ID NO: 1;

2) an amino acid sequence derived from the amino acid sequence shown in SEQ ID NO: 1 through substitution, deletion or addition of one or more amino acids; and

3) an amino acid being at least 80%, 85%, 90%, 91%, 92%, 93%, 94%, 95%, 96%, 97%, 98% or 99% identical to the amino acid sequence shown in SEQ ID NO: 1.

In the second aspect, the present disclosure provides a nucleotide sequence encoding the IL-15 analog as described above.

In the third aspect, the present disclosure provides a method for preparing an IL-15 analog, wherein the IL-15 analog as described above is expressed in a prokaryotic system.

Preferably, a nucleotide sequence encoding the IL-15 analog is linked to a prokaryotic expression vector, and transferred into prokaryotic expression host bacterium, which is induced to express the IL-15 analog.

Optionally, the IL-15 analog is expressed in the form of inclusion bodies, and the inclusion bodies are renatured.

Optionally, the inclusion bodies are dissolved in 8 M urea solution and purified by ion exchange and reverse phase chromatography.

Optionally, the prokaryotic expression vector is pET41a, and the expression host bacterium is Escherichia Coli BL21 (DE3) or C41 (DE3).

In the fourth aspect, the present disclosure provides a recombinant expression vector comprising a nucleotide sequence encoding the IL-15 analog as described above.

In the fifth aspect, the present disclosure provides a host bacterium transformed with a nucleotide sequence encoding the IL-15 analog as described above.

In the sixth aspect, the present disclosure provides a conjugate of the IL-15 analog, the amino acid sequence of the IL-15 analog comprises the amino acid sequence of IL-15, and one or more amino acids added to the C-terminal of the amino acid sequence of IL-15; and wherein the conjugate of the IL-15 analog is obtained by linking the IL-15 analog with a fatty acid chain.

Preferably, the amino acid sequence of the IL-15 analog is characterized by one of:

1) IL-15-Xₐ-Y_{b}-Z_{c}

wherein X, Y and Z each represent an amino acid sequence added at the C-terminal, and a, b and c each represent the number of the amino acids; and

wherein X and Z each are any amino acid or a combination of any amino acids, and a and c each are 0 to 20; and

wherein Y is a positively charged amino acid or a combination of any positively charged amino acids, or a combination of a positively charged amino acid and any other amino acids, and b is 1 to 7; and

2) IL-15-linker-Xₐ-Y_{b}-Z_{c}

wherein the linker represents a linker sequence between the amino acid sequence of IL-15 and the amino acid sequences added to the C-terminal thereof.

Preferably, Xₐ comprises LPBTG (SEQ ID NO: 151) with B being any amino acid, and the linker is (GS)ₙ with n being 0 to 10.

Preferably, the fatty acid chain is —(CH₂)ₘ—COOH, wherein m is 12 to 19.

Preferably, the IL-15 analog and the fatty acid chain are linked by in vitro coupling.

Preferably, the in vitro coupling comprises:

1) coupling through enzymatic reaction, wherein the fatty acid chain in the enzymatic reaction has GGG at the N-terminal;

2) coupling with free cysteine residues introduced into the IL-15 analog; and

3) coupling with the amino group at the N-terminal of the IL-15 analog.

Preferably, when coupling through enzymatic reaction, the fatty acid chain has 3 glycine residues at the N-terminal; when coupling with free cysteine residues introduced into the IL-15 analog, the fatty acid chain has a maleimide ester or a halogenated reactive group; and when coupling with the amino group at the N-terminal of the IL-15 analog, the fatty acid chain has an aldehyde group or a succinimidyl ester functional group.

Optionally, the IL-15 analog is obtained by adding a sequence comprising -GS-LPETG (SEQ ID NO: 152) to the terminal of the amino acid sequence of IL-15.

Optionally, the fatty acid chain is selected from the group consisting of:

GGG-PEG2-Lys-(CH₂)₁₆-COOH,
NHS-PEG2-PEG2-γ-Glu-(CH₂)₁₇-COOH,
GGG-PEG4-PEG4-PEG4-Lys-(CH₂)₁₇-COOH,
GGG-PEG4-γ-Glu-γ-Glu-Lys-(CH₂)₁₇-COOH,
HOOC-(CH₂)₁₆-γ-Glu-γ-Glu-Lys-GGG,
HOOC-(CH₂)₁₆-CONH-γ-Glu-γ-Glu-PEG2-Lys-Br,
GGG-γ-Glu-C2DA-2OEG-γ-Glu-(CH₂)₁₇-COOH,
GGG-γ-Glu-C2DA-2OEG-γ-Glu-(CH₂)₁₉-COOH,
GGG-OEG-C2DA-2OEG-γ-Glu-(CH₂)₁₉-COOH,
GGG-OEG-C2DA-2OEG-γ-Glu-Trx-(CH₂)₁₉-COOH,
CHO-PEG2-PEG2-γ-Glu-(CH₂)₁₇-COOH,
and
Mal-C2DA-2OEG-γ-Glu-Tn-(CH₂)₁₉-COOH.

The present inventors have found that the expression of IL-15 in Escherichia Coli could be promoted by adding some amino acids to the C-terminal of IL-15, and that positively charged amino acids could significantly enhance the expression of IL-15. The present IL-15 analog is highly expressed in Escherichia Coli, and the expression level is about 20 or even more fold higher than that of IL-15 without extra amino acids at the C-terminal. All or most of the present IL-15 analog retains the amino acid sequence of the natural wild-type IL-15, and there is no significant difference in cell activity in vitro, which lays a foundation for the industrialization of IL-15 protein drugs.

In the present disclosure, a fatty acid chain is linked with the IL-15 analog through in vitro coupling to form a coupling product of IL-15 analog-fatty acid chain. Since the fatty acid chain is a ligand of albumin and can bind to albumin in blood, the IL-15 analog-fatty acid chain coupling product entering the body will form an IL-15 analog-fatty acid chain-albumin complex. On one hand, it can increase the molecular weight to escape from the renal filtration. On the other hand, hydrolysis by intracellular lysosomes can be avoided through the binding of albumin to FcRn which mediates recycling pathway as a protection mechanism, thereby achieving a long-acting mechanism of IL-15 analog-fatty acid chain coupling product. Herein, the conjugate of the present IL-15 analog is not in the form of a drug that is co-expressed with the IL-15 receptor a subunit to form a complex as used by most domestic and foreign companies. Instead, it retains the same cellular biological pattern as native IL-15, that is, it completely retains its binding to the IL15Rα receptor to participate in signal transduction and to function in vivo.

### BRIEF DESCRIPTION OF THE DRAWINGS

FIG. 1 shows the SDS-PAGE electrophoretogram of wild-type IL-15 expressed in Escherichia Coli according to a comparative example of the present disclosure.

FIGS. 2A-2G show the SDS-PAGE electrophoretograms of IL-15 analogs expressed in Escherichia Coli according to an Example of the present disclosure, wherein

FIG. 2A shows the SDS-PAGE electrophoretograms of IL-15 analogs 1 to 3 and 5 to 8,

FIG. 2B shows the SDS-PAGE electrophoretograms of IL-15 analogs 9 to 13,

FIG. 2C shows the SDS-PAGE electrophoretograms of IL-15 analogs 4 and 14 to 18,

FIG. 2D shows the SDS-PAGE electrophoretograms of IL-15 analogs 19 to 27,

FIG. 2E shows the SDS-PAGE electrophoretograms of IL-15 analogs 28 to 33, and

FIGS. 2F and 2G show the SDS-PAGE electrophoretograms of IL-15 analogs 34 to 43, with analogs 38- to 43- representing that no specific amino acid were added to the C-terminal.

FIG. 3 shows comparison results of the expression levels of IL-15 and IL-15 analogs 1 to 33 (unit: mg/mL/10 OD).

FIG. 4 shows SDS-PAGE electrophoretograms of renatured and purified IL-15 and IL-15 analogs 11, 18, 21 and 28, wherein the results of reduced SDS-PAGE are displayed on the left side of Marker, with the reducing agent 2-mercaptoethanol added to the electrophoresis loading buffer, and the results of non-reduced SDS-PAGE are displayed on the right side of Marker, without the reducing agent 2-mercaptoethanol added to the electrophoresis loading buffer.

FIG. 5 shows the chromatogram of the purity of the conjugate of IL-15 analog in Example 3, which was determined by RP-UPLC (purity>95%).

FIG. 6 shows the molecular weight of the conjugate of IL-15 analog in Example 3, which was determined by LC-MS.

FIG. 7 shows the molecular weight of the conjugate of IL-15 analog in Example 4, which was determined by LC-MS.

FIG. 8 shows the antitumor effects of IL-15 Analog 21 and conjugate of IL-15 analog in mice in Example 7.

### DETAILED DESCRIPTION OF THE EMBODIMENTS

Hereinafter, the present invention will be further described in conjunction with examples. It should be understood that these examples are used for illustrative purposes only and are not intended to limit the protection scope of the present invention.

In the following examples, the experimental methods without special instructions were usually carried out in accordance with conventional conditions or in accordance with the conditions recommended by the manufacturer. See, for example, Sambrook et al, Molecular Cloning: A Laboratory Manual (New York: Cold Spring Harbor Laboratory Press, 1989). Unless otherwise specified, the reagents used are commercially available or publicly available reagents.

In particular embodiments according to the present disclosure, the positively charged amino acids were leucine (Lys, K), arginine (Arg, R) and histidine (His, H).

As the basis for modification, the amino acid sequence of IL-15 was selected from the group of:

1) the amino acid sequence shown in SEQ ID NO: 1;

2) an amino acid sequence derived from the amino acid sequence shown in SEQ ID NO: 1 through substitution, deletion or addition of one or more amino acids; and

3) an amino acid being at least 80%, 85%, 90%, 91%, 92%, 93%, 94%, 95%, 96%, 97%, 98% or 99% identical to the amino acid sequence shown in SEQ ID NO: 1.

In particular embodiments according to the present disclosure, the expression of IL-15, especially in prokaryotic expression systems, could be enhanced by adding one or more amino acids, especially positively charged amino acids, to the C-terminal of IL-15.

In some particular embodiments, the amino acid sequence of the IL-15 analog could be represented as the following general formula (that is, the amino acid sequence Xₐ-Y_{b}-Z_{c} was added to the C-terminal of IL-15):

IL-15-Xₐ-Y_{b}-Z_{c}

wherein X, Y and Z each represented an amino acid sequence added at the C-terminal, and a, b and c each represented the number of the amino acids; and

wherein X and Z each were any amino acid or a combination of any amino acids, and a and c each were 0 to 20; and

wherein Y was a positively charged amino acid or a combination of any positively charged amino acids, or a combination of a positively charged amino acid and any other amino acids, and b was 1 to 7.

In some particular embodiments, the amino acid sequence of the IL-15 analog could be represented as the following general formula (that is, a linker sequence and the amino acid sequence Xₐ-Y_{b}-Z_{c} were added to the C-terminal of IL-15):

IL-15-linker-Xₐ-Y_{b}-Z_{c}

wherein X, Y and Z each represented an amino acid sequence added at the C-terminal, and a, b and c each represented the number of the amino acids; and

wherein X and Z each were any amino acid or a combination of any amino acids, and a and c each were 0 to 20; and

wherein Y was a positively charged amino acid or a combination of any positively charged amino acids, or a combination of a positively charged amino acid and any other amino acids, and b was 1 to 7.

In some optional particular embodiments, the linker could be (GGGGS)ₙ, (GS)ₙ or (GAPQ)ₙ, with n being 1 to 5.

In some particular embodiments, in the above general formula of the amino acid sequence of IL-15 analogs, Xₐ comprised LPBTG (SEQ ID NO: 151), wherein B was any amino acid and the linker was (GS)ₙ, with n being 0 to 10. In a particular embodiment, the IL-15 analog was obtained by adding a sequence comprising -GS-LPETG (SEQ ID NO: 152) to the terminal of the amino acid sequence of IL-15.

In some particular embodiments, the IL-15 analogs were coupled with fatty acid chains in vitro to improve the long-term efficacy of the IL-15 analogs. The way of in vitro coupling of fatty acid chains could be selected from the group of:

1) coupling with the amino group at the N-terminal, for example, through an aldehyde group or a succinimide ester;

2) site-directed coupling with the introduced free cysteine residue (for example, introducing a free cysteine residue at the C-terminal of the IL-15 analog), through a maleimide group or a halogenated group; and

3) utilizing an enzymatic reaction, for example, the enzyme Sortase A could utilize the small peptide LPETG (SEQ ID NO: 153) at the C-terminal of IL-15 to couple the IL-15 with the fatty acid chain with GGG at the N-terminal (see M. L. Bentley et al., J. Biol. Chem. 2008, 283: 14762-14771). The fatty acid chains could comprise the structure as shown in Table 2.

TABLE 2
The structure of fatty acid chains

| Name | Fatty acid chain | Reaction with IL-15 |
|---|---|---|
| 816366 | GGG-PEG2-Lys-(CH₂)₁₆—COOH | (IL-15)-LPET-816366 |
| 823479 | GGG-PEG4-PEG4-PEG4-Lys-(CH₂)₁₇—COOH | (IL-15)-LPET-823479 |
| 823480 | GGG-PEG4-γ-Glu-γ-Glu-Lys-(CH₂)₁₇—COOH | (IL-15)-LPET-823480 |
| 818389 | HOOC—(CH₂)₁₆-γ-Glu-γ-Glu-Lys-GGG | (IL-15)-LPET-818389 |
| 817549 | HOOC—(CH₂)₁₆—CONH-γ-Glu-γ-Glu-PEG2-Lys-Br | (IL-15)-Cysteine-817549 |
| 823848 | GGG-γ-Glu-C2DA-2OEG-γ-Glu-(CH₂)₁₇—COOH | (IL-15)-LPET-823 848 |
| 823853 | GGG-γ-Glu-C2DA-2OEG-γ-Glu-(CH₂)₁₉—COOH | (IL-15)-LPET-823 853 |
| 823854 | GGG-OEG-C2DA-2OEG-γ-Glu-(CH₂)₁₉—COOH | (IL-15)-LPET-823 854 |
| 823856 | GGG-OEG-C2DA-2OEG-γ-Glu-Trx-(CH₂)₁₉—COOH | (IL-15)-LPET-823 856 |
| 820044 | NHS-PEG2-PEG2-γ-Glu-(CH₂)₁₇—COOH | 820044-(IL-15) |
| 820045 | CHO-PEG2-PEG2-γ-G1u-(CH₂)₁₇—COOH | 820045-(IL-15) |
| 823855 | Mal-C2DA-2OEG-γ-Glu-Tn-(CH₂)₁₉—COOH | (IL-15)-Cysteine-823855 |

*In the above table, LPET represents that the amino acid sequence added to the C-terminal of IL-15 includes LPET.

### Example 1: Expression of Wild-Type IL-15 and IL-15 Analogs in Escherichia Coli

1.1 Construction of Expression Vector

The wild-type IL-15 nucleotide sequence was synthesized by Sangon Biotech (Shanghai).

(1) Design of Primers

The sequence of the C-terminal of IL-15 was altered by introducing a base sequence of different amino acids to be added into the reverse primer. The amino acid sequence, the nucleotide sequence and the primer sequence (used in the construction of IL-15 analogs) of the wild-type IL-15 and constructed IL-15 analogs are shown in Table 3.

TABLE 3
Amino acid sequence, nucleotide sequence and primer sequence (used in the
construction of IL-15 analogs) of the wild-type IL-15 and IL-15 analogs

| | Sequence | SEQ ID NO: |
|---|---|---|
| IL-15 | MNWVNVISDLKKIEDLIQSMHIDATLYTESDVHPSCKVTAMK | 1 |
| | CFLLELQVISLESGDASIHDTVENLIILANNSLSSNGNVTESGC | |
| | KECEELEEKNIKEFLQSFVHIVQMFINTS | |
| IL-15 | ATGAACTGGGTGAACGTTATCAGCGACCTGAAGAAAATCGA | 2 |
| | GGATCTGATTCAGAGCATGCACATTGACGCGACCCTGTACA | |
| | CCGAAAGCGATGTGCACCCGAGCTGCAAGGTTACCGCGATG | |
| | AAATGCTTCCTGCTGGAGCTGCAAGTGATCAGCCTGGAAAG | |
| | CGGTGACGCGAGCATTCACGATACCGTTGAGAACCTGATCA | |
| | TTCTGGCGAACAACAGCCTGAGCAGCAACGGTAACGTGAC | |
| | CGAGAGCGGCTGCAAGGAATGCGAGGAACTGGAGGAAAA | |
| | GAACATCAAAGAATTCCTGCAGAGCTTTGTGCACATCGTTC | |
| | AAATGTTTATTAACACCAGC | |
| Analog | IL-15-(GS)₃-HHHHHH | 154 |
| 1 | IL-15-ggttctggttctggttctcaccaccaccaccaccac | 155 |
| | Forward primer for Analog 1: | 23 |
| | taagaaggagatatacatatgAACTGGGTGAACGTTATCAGCG | |
| | Reverse primer 1 for Analog 1: | 24 |
| | gtggtggtggtggtgagaaccagaaccagaaccGCTGGTGTTAATAAACATTT | |
| | GAACG | |
| | Reverse primer 2 for Analog 1: | 25 |
| | gtggtggtggtggtgctcgagTTAGTGGTGGTGGTGGTGGTGAGAACC | |
| Analog | IL-15-(GS)₁-HHHHHH | 156 |
| 2 | IL-15-ggttctcaccaccaccaccaccac | 157 |
| | Forward primer for Analog 2: | 26 |
| | taagaaggagatatacatatgAACTGGGTGAACGTTATCAGCG | |
| | Reverse primer 1 for Analog 2: | 27 |
| | gtggtggtggtggtggtgagaaccGCTGGTGTTAATAAACATTTGAACG | |
| | Reverse primer 2 for Analog 2: | 28 |
| | gtggtggtggtggtgctcgagTTAGTGGTGGTGGTGGTGGTGAG | |
| Analog | IL-15-PLASTKKR | 158 |
| 3 | IL-15-ccacttgctagcaccaaaaagcgt | 159 |
| | Forward primer for Analog 3: | 29 |
| | taagaaggagatatacatatgAACTGGGTGAACGTTATCAGCG | |
| | Reverse primer 1 for Analog 3: | 30 |
| | acgctttttggtgctagcaagtggGCT GGT GTTAATAAAC ATTT GAACG | |
| | Reverse primer 2 for Analog 3: | 31 |
| | gtggtggtggtggtgctcgagTTAACGCTTTTTGGTGCTAGCAAG | |
| Analog | IL-15-LPKSAKKK | 160 |
| 4 | IL-15-cttccaaagtctgctaaaaagaag | 161 |
| | Forward primer for Analog 4: | 32 |
| | taagaaggagatatacatatgAACTGGGTGAACGTTATCAGCG | |
| | Reverse primer 1 for Analog 4: | 33 |
| | cttctttttagcagactttggaagGCTGGTGTTAATAAACATTTGAACG | |
| | Reverse primer 2 for Analog 4: | 34 |
| | gtggtggtggtggtgctcgagTTACTTCTTTTTAGCAGACTTTGGAAGG | |
| Analog | IL-15-KKKKKKK | 162 |
| 5 | IL-15-aaaaagaagaaaaaaaagaag | 163 |
| | Forward primer for Analog 5: | 35 |
| | taagaaggagatatacatatgAACTGGGTGAACGTTATCAGCG | |
| | Reverse primer 1 for Analog 5: | 36 |
| | cttcttttttttcttctttttGCTGGTGTTAATAAACATTTGAACG | |
| | Reverse primer 2 for Analog 5: | 37 |
| | gtggtggtggtggtgctcgagTTACTTCTTTTTTTTCTTCTTTTTGCTGG | |
| Analog | IL-15-(GAPQGAPQ)-LVESAHHH | 164 |
| 6 | IL-15-ggtgctccacagggtgctccacagcttgtggaatctgcacaccatcat | 165 |
| | Forward primer for Analog 6: | 38 |
| | taagaaggagatatacatatgAACTGGGTGAACGTTATCAGCG | |
| | Reverse primer 1 for Analog6: | 39 |
| | attccacaagctgtggagcaccctgtggagcaccGCTGGTGTTAATAAACATTT | |
| | GAACG | |
| | Reverse primer 2 for Analog 6: | 40 |
| | gtggtggtggtggtgctcgagTTAatgatggtgtgcagATTCCACAAGCTGTGG | |
| | AGCAC | |
| Analog | IL-15-GS-LVSSAHHK | 166 |
| 7 | IL-15-ggtagccttgtatctagcgctcaccacaaa | 167 |
| | Forward primer for Analog 7: | 41 |
| | taagaaggagatatacatatgAACTGGGTGAACGTTATCAGCG | |
| | Reverse primer 1 for Analog 7: | 42 |
| | tttgtggtgagcgctagatacaaggctaccGCTGGTGTTAATAAACATTTGAA | |
| | CG | |
| | Reverse primer 2 for Analog 7: | 43 |
| | gtggtggtggtggtgctcgagTTATTTGTGGTGAGCGCTAGATACAA | |
| Analog | IL-15-GS-LIEHHRRK | 168 |
| 8 | IL-15-ggtagccttatcgaacaccaccgtcgcaaa | 169 |
| | Forward primer for Analog 8: | 44 |
| | taagaaggagatatacatatgAACTGGGTGAACGTTATCAGCG | |
| | Reverse primer 1 for Analog 8: | 45 |
| | tttgcgacggtggtgttcgataaggctaccGCTGGTGTTAATAAACATTTGAA | |
| | CG | |
| | Reverse primer 2 for Analog 8: | 46 |
| | gtggtggtggtggtgctcgagTTATTTGCGACGGTGGTGTTCG | |
| Analog | IL-15-GS-IVEHRKKK | 170 |
| 9 | IL-15-ggtagcattgtagaacaccgtaagaaaaag | 171 |
| | Forward primer for Analog 9: | 47 |
| | taagaaggagatatacatatgAACTGGGTGAACGTTATCAGCG | |
| | Reverse primer 1 for Analog 9: | 48 |
| | ctttttcttacggtgttctacaatgctaccGCTGGTGTTAATAAACATTTGAACG | |
| | Reverse primer 2 for Analog 9: | 49 |
| | gtggtggtggtggtgctcgagTTACTTTTTCTTACGGTGTTCTACAATGC | |
| Analog | IL-15-GS-VPKTGRRR | 172 |
| 10 | IL-15-ggtagcgtaccaaaaactggtcgtcgccgt | 173 |
| | Forward primer for Analog 10: | 50 |
| | taagaaggagatatacatatgAACTGGGTGAACGTTATCAGCG | |
| | Reverse primer 1 for Analog 10: | 51 |
| | acggcgacgaccagtttttggtacgctaccGCTGGTGTTAATAAACATTTGAA | |
| | CG | |
| | Reverse primer 2 for Analog 10: | 52 |
| | gtggtggtggtggtgctcgagTTAACGGCGACGACCAGTTTTT | |
| Analog | IL-15-GS-LVASGKK | 174 |
| 11 | IL-15-ggtagcctggttgctagcggtaaaaag | 175 |
| | Forward primer for Analog 11: | 53 |
| | taagaaggagatatacatatgAACTGGGTGAACGTTATCAGCG | |
| | Reverse primer 1 for Analog 11: | 54 |
| | ctttttaccgctagcaaccaggctaccGCTGGTGTTAATAAACATTTGAACG | |
| | Reverse primer 2 for Analog 11: | 55 |
| | gtggtggtggtggtgctcgagTTACTTTTTACCGCTAGCAACCAGG | |
| Analog | IL-15-GS-HRKSGHHH | 176 |
| 12 | IL-15-ggtagccatcgtaaatctggtcaccatcat | 177 |
| | Forward primer for Analog 12: | 56 |
| | taagaaggagatatacatatgAACTGGGTGAACGTTATCAGCG | |
| | Reverse primer 1 for Analog 12: | 57 |
| | atgatggtgaccagatttacgatggctaccGCTGGTGTTAATAAACATTTGAA | |
| | CG | |
| | Reverse primer 2 for Analog 12: | 58 |
| | gtggtggtggtggtgctcgagTTAATGATGGTGACCAGATTTACGATG | |
| Analog | IL-15-GS-LPKTGRHK | 178 |
| 13 | IL-15-ggtagccttccaaaaactggtcgtcacaag | 179 |
| | Forward primer for Analog 13: | 59 |
| | taagaaggagatatacatatgAACTGGGTGAACGTTATCAGCG | |
| | Reverse primer 1 for Analog 13: | 60 |
| | cttgtgacgaccagtttttggaaggctaccGCTGGTGTTAATAAACATTTGAA | |
| | CG | |
| | Reverse primer 2 for Analog 13: | 61 |
| | gtggtggtggtggtgctcgagTTACTTGTGACGACCAGTTTTTGGA | |
| Analog | IL-15-KKKTGRRH | 180 |
| 14 | IL-15-aaaaagaagactggtcgtcgccat | 181 |
| | Forward primer for Analog 14: | 62 |
| | taagaaggagatatacatatgAACTGGGTGAACGTTATCAGCG | |
| | Reverse primer 1 for Analog 14: | 63 |
| | atggcgacgaccagtcttctttttGCTGGTGTTAATAAACATTTGAACG | |
| | Reverse primer 2 for Analog 14: | 64 |
| | gtggtggtggtggtgctcgagTTAATGGCGACGACCAGTCTTCTT | |
| Analog | IL-15-LPRSGRHK | 182 |
| 15 | IL-15-cttccacgttctggtcgtcataag | 183 |
| | Forward primer for Analog 15: | 65 |
| | taagaaggagatatacatatgAACTGGGTGAACGTTATCAGCG | |
| | Reverse primer 1 for Analog 15: | 66 |
| | cttatgacgaccagaacgtggaagGCTGGTGTTAATAAACATTTGAACG | |
| | Reverse primer 2 for Analog 15: | 67 |
| | gtggtggtggtggtgctcgagTTACTTATGACGACCAGAACGTGGA | |
| Analog | IL-15-LVETHHHH | 184 |
| 16 | IL-15-ctggttgaaactcaccatcatcac | 185 |
| | Forward primer for Analog 16: | 68 |
| | taagaaggagatatacatatgAACTGGGTGAACGTTATCAGCG | |
| | Reverse primer 1 for Analog 16: | 69 |
| | gtgatgatggtgagtttcaaccagGCTGGTGTTAATAAACATTTGAACG | |
| | Reverse primer 2 for Analog 16: | 70 |
| | gtggtggtggtggtgctcgagTTAGTGATGATGGTGAGTTTCAACCAG | |
| Analog | IL-15-VRPETHHH | 186 |
| 17 | IL-15-gttcgtccagaaactcaccatcat | 187 |
| | Forward primer for Analog 17: | 71 |
| | taagaaggagatatacatatgAACTGGGTGAACGTTATCAGCG | |
| | Reverse primer 1 for Analog 17: | 72 |
| | atgatggtgagtttctggacgaacGCT GGT GTTAATAAACATTTGAACG | |
| | Reverse primer 2 for Analog 17: | 73 |
| | gtggtggtggtggtgctcgagTTAATGATGGTGAGTTTCTGGACGAA | |
| Analog | IL-15-KKK | 188 |
| 18 | IL-15-aaaaaaaag | 189 |
| | Forward primer for Analog 18: | 74 |
| | taagaaggagatatacatatgAACTGGGTGAACGTTATCAGCG | |
| | Reverse primer 1 for Analog 18: | 75 |
| | cttttttttGCTGGTGTTAATAAACATTTGAACG | |
| | Reverse primer 2 for Analog 18: | 76 |
| | gtggtggtggtggtgctcgagTTACTTTTTTTTGCTGGTGTTAATAAACA | |
| Analog | IL-15-RHHHH | 190 |
| 19 | IL-15-cgtcaccatcatcat | 191 |
| | Forward primer for Analog 19: | 77 |
| | taagaaggagatatacatatgAACTGGGTGAACGTTATCAGCG | |
| | Reverse primer 1 for Analog 19: | 78 |
| | atgatgatggtgacgGCTGGTGTTAATAAACATTTGAACG | |
| | Reverse primer 2 for Analog 19: | 79 |
| | gtggtggtggtggtgctcgagTTAATGATGATGGTGACGGCTGG | |
| Analog | IL-15-KRETHHHH | 192 |
| 20 | IL-15-aagcgtgaaactcaccatcatcat | 193 |
| | Forward primer for Analog 20: | 80 |
| | taagaaggagatatacatatgAACTGGGTGAACGTTATCAGCG | |
| | Reverse primer 1 for Analog 20: | 81 |
| | atgatgatggtgagtttcacgcttGCTGGTGTTAATAAACATTTGAACG | |
| | Reverse primer 2 for Analog 20: | 82 |
| | gtggtggtggtggtgctcgagTTAATGATGATGGTGAGTTTCACGCT | |
| Analog | IL-15-GS-LPETG-GSGGSHHHHHH | 194 |
| 21 | IL-15-ggttctctgccggaaaccggtggttctggtggttctcaccaccaccaccaccac | 195 |
| | Forward primer for Analog 21: | 83 |
| | taagaaggagatatacatatgAACTGGGTGAACGTTATCAGCG | |
| | Reverse primer 1 for Analog 21: | 84 |
| | accaccagaaccaccggtttccggcagagaaccGCTGGTGTTAATAAACATT | |
| | Reverse primer 2 for Analog 21: | 85 |
| | gtggtggtggtggtgctcgagTTAGTGGTGGTGGTGGTGGTGAGAACC | |
| | ACCAGAACC | |
| Analog | IL-15-HLETGKKK | 196 |
| 22 | IL-15-caccttgaaactggtaaaaagaag | 197 |
| | Forward primer for Analog 22: | 86 |
| | taagaaggagatatacatatgAACTGGGTGAACGTTATCAGCG | |
| | Reverse primer 1 for Analog 22: | 87 |
| | cttctttttaccagtttcaaggtgGCTGGTGTTAATAAACATTTGAACG | |
| | Reverse primer 2 for Analog 22: | 88 |
| | gtggtggtggtggtgctcgagTTACTTCTTTTTACCAGTTTCAAGGTGG | |
| Analog | IL-15-HVESGRRR | 198 |
| 23 | IL-15-catgttgaatctggtcgtcgccgt | 199 |
| | Forward primer for Analog 23: | 89 |
| | taagaaggagatatacatatgAACTGGGTGAACGTTATCAGCG | |
| | Reverse primer 1 for Analog 23: | 90 |
| | acggcgacgaccagattcaacatgGCTGGTGTTAATAAACATTTGAACG | |
| | Reverse primer 2 for Analog 23: | 91 |
| | gtggtggtggtggtgctcgagTTAACGGCGACGACCAGATTCA | |
| Analog | IL-15-RRHTGKKK | 200 |
| 24 | IL-15-cgtcgtcatactggtaaaaagaag | 201 |
| | Forward primer for Analog 24: | 92 |
| | taagaaggagatatacatatgAACTGGGTGAACGTTATCAGCG | |
| | Reverse primer 1 for Analog 24: | 93 |
| | cttctttttaccagtatgacgacgGCTGGTGTTAATAAACATTTGAACG | |
| | Reverse primer 2 for Analog 24: | 94 |
| | gtggtggtggtggtgctcgagTTACTTCTTTTTACCAGTATGACGACGG | |
| Analog | IL-15-HVKTGHHH | 202 |
| 25 | IL-15-catgttaagactggtcaccatcat | 203 |
| | Forward primer for Analog 25: | 95 |
| | taagaaggagatatacatatgAACTGGGTGAACGTTATCAGCG | |
| | Reverse primer 1 for Analog 25: | 96 |
| | atgatggtgaccagtcttaacatgGCTGGTGTTAATAAACATTTGAACG | |
| | Reverse primer 2 for Analog 25: | 97 |
| | gtggtggtggtggtgctcgagTTAATGATGGTGACCAGTCTTAACATGG | |
| Analog | IL-15-HVKSGRHH | 204 |
| 26 | IL-15-catgttaagtctggtcgtcatcat | 205 |
| | Forward primer for Analog 26: | 98 |
| | taagaaggagatatacatatgAACTGGGTGAACGTTATCAGCG | |
| | Reverse primer 1 for Analog 26: | 99 |
| | atgatgacgaccagacttaacatgGCTGGTGTTAATAAACATTTGAACG | |
| | Reverse primer 2 for Analog 26: | 100 |
| | gtggtggtggtggtgctcgagTTAATGATGACGACCAGACTTAACATGG | |
| Analog | IL-15-HVKSSHRH | 206 |
| 27 | IL-15-catgttaagtctagccatcgtcac | 207 |
| | Forward primer for Analog 27: | 101 |
| | taagaaggagatatacatatgAACTGGGTGAACGTTATCAGCG | |
| | Reverse primer 1 for Analog 27: | 102 |
| | gtgacgatggctagacttaacatgGCTGGTGTTAATAAACATTTGAACG | |
| | Reverse primer 2 for Analog 27: | 103 |
| | gtggtggtggtggtgctcgagTTAGTGACGATGGCTAGACTTAACATGG | |
| Analog | IL-15-(GS)₅-LVKSGHHH | 208 |
| 28 | IL-15-ggtagcggtagcggtagcggtagcggtagcctggtaaagtctggtcaccatcat | 209 |
| | Forward primer for Analog 28: | 104 |
| | taagaaggagatatacatatgAACTGGGTGAACGTTATCAGCG | |
| | Reverse primer 1 for Analog 28: | 105 |
| | gctaccgctaccgctaccgctaccgctaccGCTGGTGTTAATAAACATTTGAA | |
| | CG | |
| | Reverse primer 2 for Analog 28: | 106 |
| | atgatggtgaccagactttaccagGCTACCGCTACCGCTACCG | |
| | Analog 28 Reverse primer for 3: | 107 |
| | gtggtggtggtggtgctcgagTTAATGATGGTGACCAGACTTTACCAG | |
| Analog | IL-15-RPKSGHHK | 210 |
| 29 | IL-15-cgtccaaagagcggtcaccataag | 211 |
| | Forward primer for Analog 29: | 108 |
| | taagaaggagatatacatatgAACTGGGTGAACGTTATCAGCG | |
| | Reverse primer 1 for Analog 29: | 109 |
| | cttatggtgaccgctctttggacgGCTGGTGTTAATAAACATTTGAACG | |
| | Reverse primer 2 for Analog 29: | 110 |
| | gtggtggtggtggtgctcgagTTACTTATGGTGACCGCTCTTTGGA | |
| Analog | IL-15-KKC | 212 |
| 30 | IL-15-aaaaagtgt | 213 |
| | Forward primer for Analog 30: | ill |
| | taagaaggagatatacatatgAACTGGGTGAACGTTATCAGCG | |
| | ReverseprimerforAnalog30: | 112 |
| | gtggtggtggtggtgctcgagTTAacactttttGCTGGTGTTAATAAACATTTG | |
| | AACG | |
| Analog | IL-15-LHKAGKHH | 214 |
| 31 | IL-15-cttcacaaggctggtaaacaccat | 215 |
| | Forward primer for Analog 31: | 113 |
| | taagaaggagatatacatatgAACTGGGTGAACGTTATCAGCG | |
| | Reverse primer 1 for Analog 31: | 114 |
| | atggtgtttaccagccttgtgaagGCTGGTGTTAATAAACATTTGAACG | |
| | Reverse primer 2 for Analog 31: | 115 |
| | gtggtggtggtggtgctcgagTTAATGGTGTTTACCAGCCTTGTGAA | |
| Analog | IL-15-K | 216 |
| 32 | IL-15-aaa | 217 |
| | Forward primer for Analog 32: | 116 |
| | taagaaggagatatacatatgAACTGGGTGAACGTTATCAGCG | |
| | Reverse primer for Analog 32: | 117 |
| | gtggtggtggtggtgctcgagTTAtttGCTGGTGTTAATAAACATTTGAAC | |
| | G | |
| Analog | IL-15-KK | 218 |
| 33 | IL-15-aaaaag | 219 |
| | Forward primer for Analog 33: | 118 |
| | taagaaggagatatacatatgAACTGGGTGAACGTTATCAGCG | |
| | Reverse primer for Analog 33: | 119 |
| | gtggtggtggtggtgctcgagTTActttttGCTGGTGTTAATAAACATTTGAA | |
| | CG | |

The analogs 34 to 43 were formed by introducing mutations into the wild-type IL-15 to form IL-15 mutants, and then adding the sequence GSLPETGGGSGGSHHHHHH (SEQ ID NO: 220) to the C-terminals based on the IL-15 mutants. The analogs 34- to 43- were the mutants of IL-15 formed just after the mutations were introduced, without further adding the sequence GSLPETGGGSGGSHHHHHH (SEQ ID NO: 220) to the C-terminals. The nucleotide sequences of the mutated analogs 34 to 43 and analogs 38- to 43- without adding the sequence GSLPETGGGSGGSHHHHHH (SEQ ID NO: 220) to the C-terminals were synthesized by the Genewiz, Inc., and cloned into the pET41a vectors.

TABLE 4
Analogs 34-43

| | | SEQ | Identity | SEQ ID NO: |
|---|---|---|---|---|
| | | ID | with IL- | (Amino Acid |
| Name | Sequence of Protein | NO: | 15 | Sequence) |
| IL-15 | MNWVNVISDLKKIEDLIQSMHIDATLYTESDVH | 1 | | 2 |
| | PSCKVTAMKCFLLELQVISLESGDASIHDTVEN | | | |
| | LIILANNSLSSNGNVTESGCKECEELEEKNIKEF | | | |
| | LQSFVHIVQMFINTS | | | |
| Analog | MNWVNVISDLKKIEDLIQSMHIDATLYTESDVH | 3 | 99% | 13 |
| 34 | PSCKVTAMKCFLLELQVISLESGDASIHDTVEN | | | |
| | LIILANNSLSSNANVTESGCKECEELEEKNIKEF | | | |
| | LQSFVHIVQMFINTSGSLPETGGSGGSHHHHHH | | | |
| Analog | MANWVNVISDLKKIEDLIQSMHIDATLYTESDV | 4 | 97% | 14 |
| 35 | HPSCKVTAMKCFLLELQVISLESGDASIHDTVE | | | |
| | NLIILANNSLSSNANVTESGCKECEELEEKNIKE | | | |
| | FLQSFVHIVQMFINTSGSLPETGGSGGSHHHHH | | | |
| | H | | | |
| Analog | MNWVNVISDLKKIEDLIQSMHIRGDLYTESDV | 5 | 96% | 15 |
| 36 | HPSCKVTAMKCFLLELQVISLESGDASIHDTVE | | | |
| | NLIILANNSLSSNANVTESGCKECEELEEKNIKE | | | |
| | FLQSFVHIVQMFINTSGSLPETGGSGGSHHHHH | | | |
| | H | | | |
| Analog | MSNWVNVISDLKKIEDLIQSVHIRGDLYTESDV | 6 | 93% | 16 |
| 37 | HPSCKVTAMKCFLLELQVISLESGDGSIHDTVE | | | |
| | NLIILAQQSLSSNANVTESGCKECEELEEKNIKE | | | |
| | FLQSFVHIVQMFINTSGSLPETGGSGGSHHHHH | | | |
| | H | | | |
| Analog | MSNWVNVISDLKKIEDLIQSVHIRGDLYTESDV | 7 | 90% | 17 |
| 38 | HPSCKVTAMKCFLLELQVISLESGDGSIHDTVE | | | |
| | NLIILAQQSLSSNANVTESGCKECEELSEKNIKE | | | |
| | FLQSFVHIVQVFINTSGSLPETGGSGGSHHHHH | | | |
| | H | | | |
| Analog | MSNWVNVISDLRKIRGDIQSVHIDATLYTESDV | 8 | 90% | 18 |
| 39 | HPSCRVTAMKCFLLELQVISLESGDASIHDTVE | | | |
| | NLIILANQSLSSNANVTESGCKECEELEEKNIKE | | | |
| | FLQSFVHIVQLFIQTSGSLPETGGSGGSHHHHH | | | |
| | H | | | |
| Analog | MSNWVNVISDLRKIRGDLNAVHVDATLYTESD | 9 | 85% | 19 |
| 40 | VHPSCRVTAMKCFLLELQVISLESGDASIHDTV | | | |
| | ENLIILANQSLSSNANVTESGCKECEELEEKNIK | | | |
| | EFLQSFVHIVQLFIQTSGSLPETGGSGGSHHHH | | | |
| | HH | | | |
| Analog | MSNWVNVISDLRKIRGDIQSVHIDATLYTESDV | 10 | 85% | 20 |
| 41 | HPSCRVTAMKCFLLELQVISLESGDASIHDTVE | | | |
| | NLIILANQSLAAQAQLTESGCKECEELEEKNIKE | | | |
| | FLQSFVHIVQLFIQTSGSLPETGGSGGSHHHHH | | | |
| | H | | | |
| Analog | MSNWVNVISDLRKIRGDLNAVHVDATLYTESD | 11 | 80% | 21 |
| 42 | VHPSCRVTAMKCFLLELQVISLESGDASIHDTV | | | |
| | ENLIILANQSLAAQAQLTESGCKECEELEEKNIK | | | |
| | EFLQSFVHIVQLFIQTSGSLPETGGSGGSHHHH | | | |
| | HH | | | |
| Analog | MSNWVNVISDLRKIRGDIQSVHIDATLYTESDV | 12 | 80% | 22 |
| 43 | HPSCRVTAMKCFLLELQLISLDSGDASIHETVE | | | |
| | QLILLANQSLAAQAQLTESGCKECEELEEKNIK | | | |
| | EFLQSFVHIVQLFIQTSGSLPETGGSGGSHHHH | | | |
| | HH | | | |

(2) PCR Amplification

The PCR amplification was classified into three cases as follows.

In the first case, only one reverse primer was required, and a total of 1 run of PCR was performed.

Sample loading system: 50 μL (1 tube)

Primers: forward primer and reverse primer

Template: IL-15

Annealing temperature: 61° C.

Extension time: 30 s

In the second case, two reverse primers were required, and a total of 2 runs of PCR were performed.

1^{st} Run of PCR

Sample loading system: 20 μL (1 tube)

Primers: forward primer and reverse primer 1

Template: IL-15

Annealing temperature: 61° C.

Extension time: 30 s

2^{nd} run of PCR

Sample loading system: 50 μL (1 tube)

Primers: forward primer and reverse primer 2

Template: PCR product of 1^{st} run

Annealing temperature: 61° C.

Extension time: 30 s

In the third case, three reverse primers were required, and a total of 3 runs of PCR were performed.

1^{st} run of PCR

Sample loading system: 20 μL (1 tube)

Primers: forward primer and reverse primer 1

Template: IL-15

Annealing temperature: 61° C.

Extension time: 30 s

2^{nd} run of PCR

Sample loading system: 20 μL (1 tube)

Primers: forward primer and reverse primer 2

Template: PCR product of 1^{st} run

Annealing temperature: 61° C.

Extension time: 30 s

3^{rd} run of PCR

Sample loading system: 50 μL (1 tube)

Primers: forward primer and reverse primer 3

Template: PCR product of 2^{nd} run

Annealing temperature: 61° C.

Extension time: 30 s

Sample Loading System for PCR: 20 μL

| | | |
|---|---|---|
| 5 × TransStart ® FastPfu Fly buffer | 4 | μL |
| Forward primer (5 μM) | 1.2 | μL |
| Reverse primer (5 μM) | 1.2 | μL |
| dNTPs (2.5 mM) | 1.6 | μL |
| Template | 0.5 | μL |
| Pfu DNA polymerase | 0.4 | μL |

| | |
|---|---|
| ddH₂O | making up to 20 μL |

Sample Loading System for PCR: 50 μL

| | | |
|---|---|---|
| 5 × TransStart ® FastPfu Fly buffer | 10 | μL |
| Forward primer (5 μM) | 3 | μL |
| Reverse primer (5 μM) | 3 | μL |
| dNTPs (2.5 mM) | 4 | μL |
| Template | 0.5 | μL |
| Pfu DNA polymerase | 1 | μL |

| | |
|---|---|
| ddH₂O | making up to 50 μL |

Amplification Procedure for PCR:

| | | | |
|---|---|---|---|
| 98° C. | | 5 | min |
| 98° C. | | 20 | s |
| | {close oversize brace} | | |
| 61° C. | | 20 | s |
| 72° C. | | 30 | s |
| 72° C. | | 10 | min |

(3) Enzyme Digestion of Vector

Reaction system: 30 μL, incubating at 37° C. overnight after mixing

| | |
|---|---|
| pET41a vector | 5 μg |
| NdeI | 1 μL |
| XhoI | 1 μL |
| 10 × H buffer | 3 μL |
| ddH₂O | making up to 30 μL |

(4) PCR Products and Digested Vectors in the Gels were Recovered.

(5) Loading of Recombinants

Reaction system: 20 μL

| | | |
|---|---|---|
| Digested pET41a vector | 13.5 | μL |
| PCR product | 0.5 | μL |
| 5 × CE II buffer | 4 | μL |
| Exnase II | 2 | μL |

The molar ratio of vector to fragment was 2:1. After reacting at 37° C. for 30 min, it was immediately cooled on ice and transformed into DH5α.

(6) Sterility Test

0.5 mL of Amp-resistant LB media was added to a 1.5 mL centrifuge tube and well-grown single colonies were inoculated thereto. A total of 5 tubes were inoculated and they were incubated in a shaker under shaking at 37° C. After incubation for 3 h, 1 μL of the culture was taken as a template for PCR of the bacterial suspension.

Reaction system for PCR: 10 μL

| | | |
|---|---|---|
| 10 × Taq DNA polymerase buffer | 1 | μL |
| T7-P (5 μM) | 1 | μL |
| T7-T (5 μM) | 1 | μL |
| dNTPs (2.5 mM) | 0.8 | μL |
| Template | 1 | μL |
| Taq DNA polymerase | 0.1 | μL |
| ddH₂O | 5.1 | μL |

Amplification Procedure for PCR:

| | | | |
|---|---|---|---|
| 94° C. | | 5 | min |
| 94° C. | | 30 | s |
| | {close oversize brace} | | |
| 55° C. | | 30 | s |
| 72° C. | | 1 | min |
| 72° C. | | 5 | min |

After PCR was completed, it was detected by agarose gel electrophoresis. Three positive clones were selected for sequencing (Sangon Biotech).

(7) Preservation of Positive Bacterial Suspension and Extraction of Plasmids

The positive clones with correct sequencing were inoculated into 5 mL of Amp-resistant LB liquid media with an inoculation volume of 10 μL. 500 μL of bacterial suspension was added into a 1.5 mL centrifuge tube. 500 μL of 40% glycerol was added for storage. It was labeled with the name, host bacteria and date, capped and stored in a refrigerator at −80° C.

The remaining bacterial suspension was collected by centrifugation for plasmid extraction.

1.2 Protein Expression of Wild-Type IL-15 and IL-15 Analogs

(1) Transformation of BL21 (DE3)

a) 2 μL of plasmid was added to 100 μL of BL21 (DE3) competent cells, and it was mixed immediately and placed on ice for 30 min.

b) It was heat-shocked at 42° C. for 90 s, followed by a rapid ice bath for 2 min.

c) 500 μL of LB media was added, then it was incubated at 37° C. under shaking (≤200 rpm) for 60 minutes.

d) It was centrifuged at 6000 rpm for 1 min. Most of the supernatant was discarded, and about 100 to 150 μL of the supernatant was retained. After the pellet was resuspended, they were spread on LB plates containing Amp and cultured at 37° C. overnight.

(2) Small-Scale Expression

a) Bacteria preservation: One single clone was picked into 1 mL of Amp-resistant LB media. It was incubated at 37° C. under shaking at 220 rpm for about 5 h. 1 mL of 40% glycerol was added. It was divided into 2 tubes and cryopreserved at −80° C.

b) 2.5 mL of LB liquid media containing Amp was added to the tube in the previous step. The culture was incubated at 37° C. under shaking at 220 rpm overnight.

c) The bacterial suspension incubated overnight was inoculated into 20 mL of LB media containing Amp at a ratio of 1:50. It was incubated at 37° C. under shaking at 220 rpm to reach OD600=0.6 (about 3 h). IPTG was added at a final concentration of 0.5 mM. It was incubated at 37° C. under shaking at 220 rpm for 3 h.

(3) Expression Level Determination by SDS-PAGE

a) The cultural suspension was determined for OD600. 10 OD bacterial suspension was taken, and centrifuged at 10000 rpm for 2 min. The supernatant was removed.

b) The pellet was resuspended with 1 mL of lysis buffer (10 mM Tris-HCl, pH 8.0) placed on ice and lysed by ultrasonication. Ultrasonic conditions: 130 W, 4 min, on 3 s, off 3 s.

c) After ultrasonication, 80 μL was taken and labeled as “T (total)”. The remaining liquid was centrifuged at 12000 rpm for 10 min to obtain “S (supernatant)” and “P (pellet)”. 20 μL of 5×Reducing Loading Buffer was added to 80 μL of T, P and S, respectively. Then they were heated at 95° C. for 5 min and 12.5 μL (0.1 OD) of each sample was taken for SDS-PAGE electrophoresis.

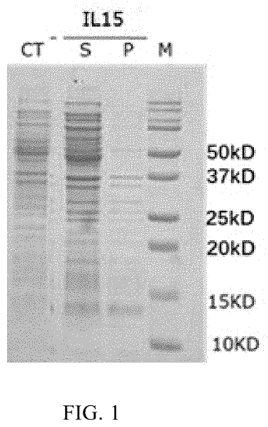

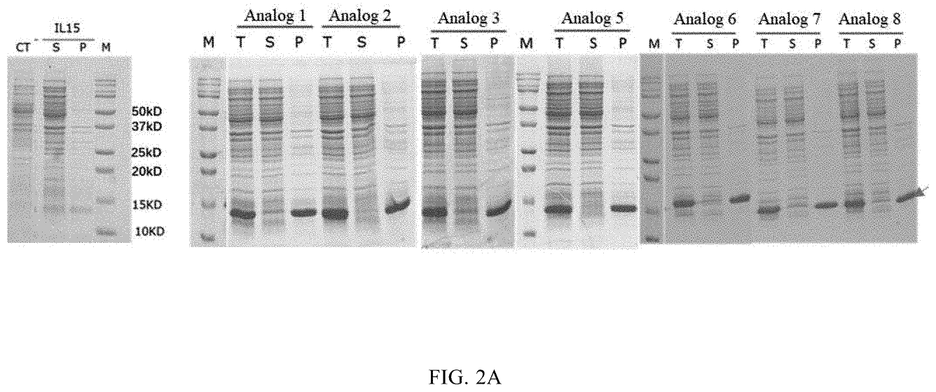

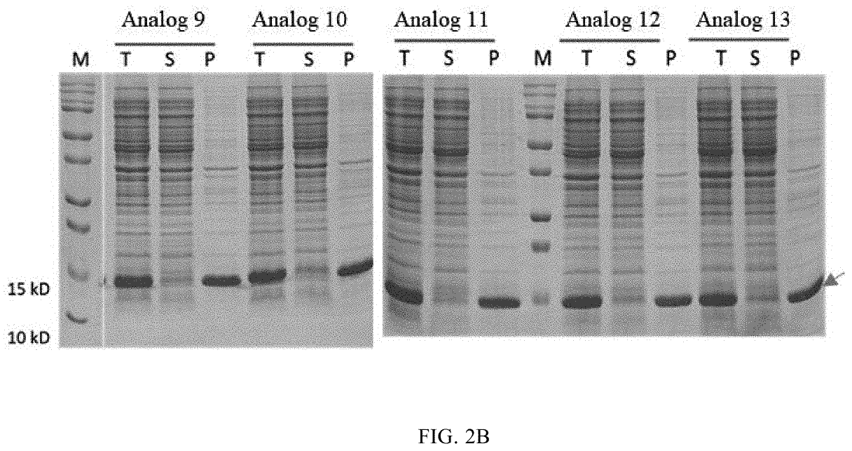

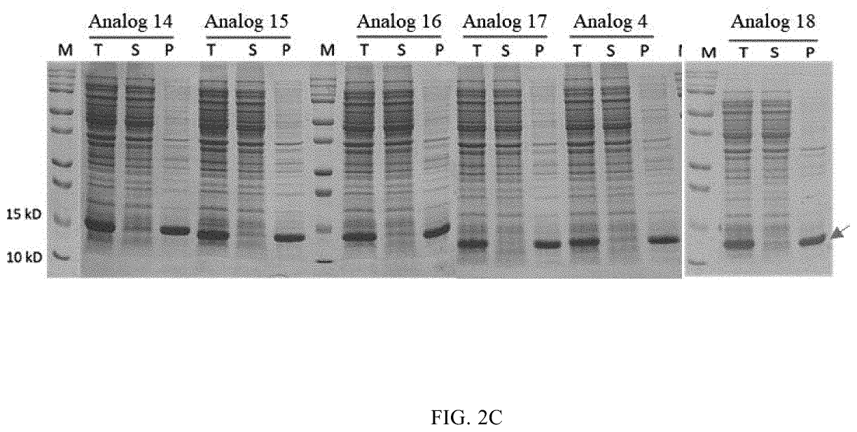

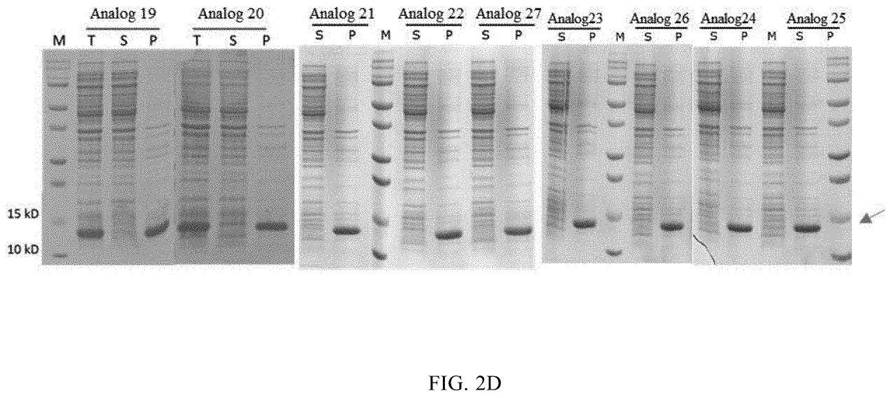

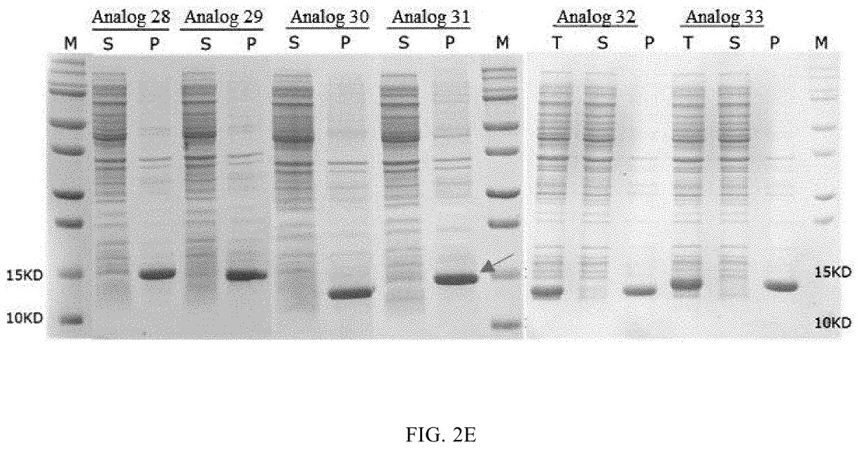

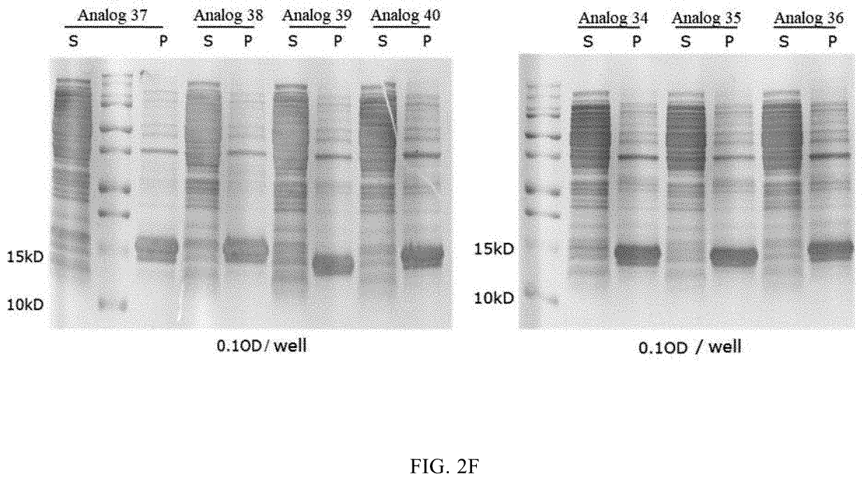

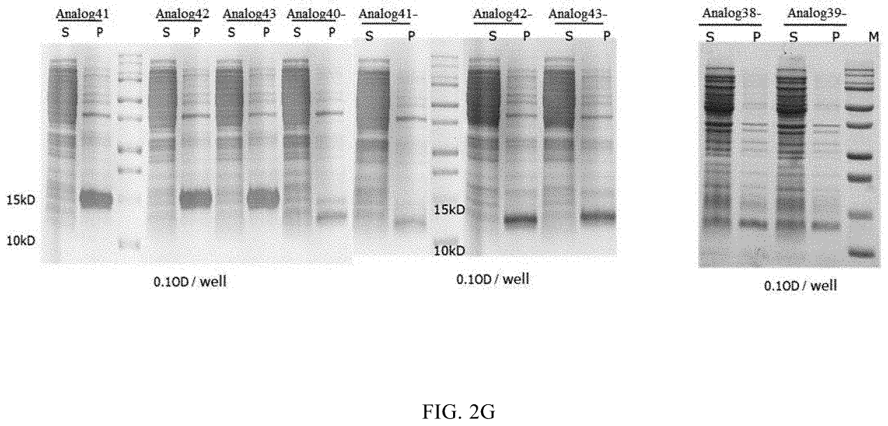

The electrophoretogram of wild-type IL-15 was shown in FIG. 1, wherein the expression of wild-type IL-15 was virtually undetectable in “T (total)”, “S (supernatant)” or “P (pellet)”. The electrophoretograms of IL-15 analogs 1 to 33 were shown in FIGS. 2A-2E, wherein the expression levels of the IL-15 analogs were significantly higher than that of wild-type IL-15. Further, the expression levels of the IL-15 analogs (analogs 34 to 43) with the sequence GSLPETGGGSGGSHHHHHH (SEQ ID NO: 220) at the C-terminal were also significantly higher than those of the IL-15 analogs (analogs 38- to 43-) without the sequence GSLPETGGGSGGSHHHHHH (SEQ ID NO: 220) at the C-terminal, as shown in FIGS. 2F and 2G.

(4) Expression Level Determination by HPLC

a) After expression, 10 OD cells were collected and resuspended with 1 mL of 10 mM Tris-HCl buffer at pH 8.0.

b) Sonication was performed under the same conditions as those for running gel electrophoresis as described in the above step (3).

c) After sonication, it was centrifuged at 12000 rpm for 10 min. The supernatant was discarded.

d) The pellet was added with 1 mL of freshly prepared 8 M Urea/10 mM Tris-HCl (pH 8.0, 10 mM DTT) and dissolved by shaking at room temperature for about 1 h.

e) It was filtered with a 0.2 μm filter and loaded for HPLC analysis. The analysis was performed using a C4 analytical column with 0.1% TFA in deionized water as mobile phase A and 0.1% TFA in acetonitrile as mobile phase B, followed by a 15-minute gradient from 20% B to 60% B.

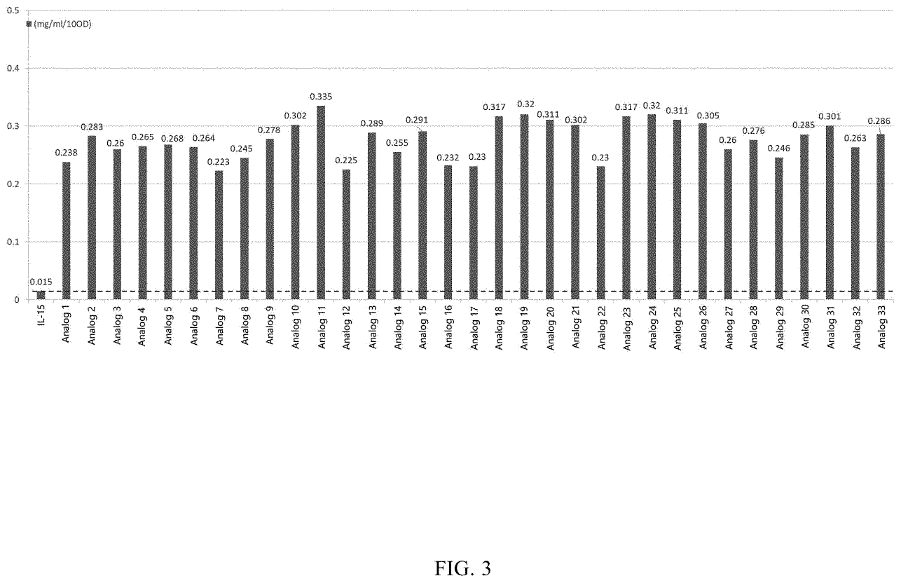

As shown by the HPLC quantitative results in FIG. 3, the expression levels of the IL-15 analogs 1 to 33 were about 20-fold higher than that of the wild-type IL-15.

### Example 2: In Vitro Cell Activity Assay of IL-15 Analogs

2.1 Preparation of IL-15 Analogs

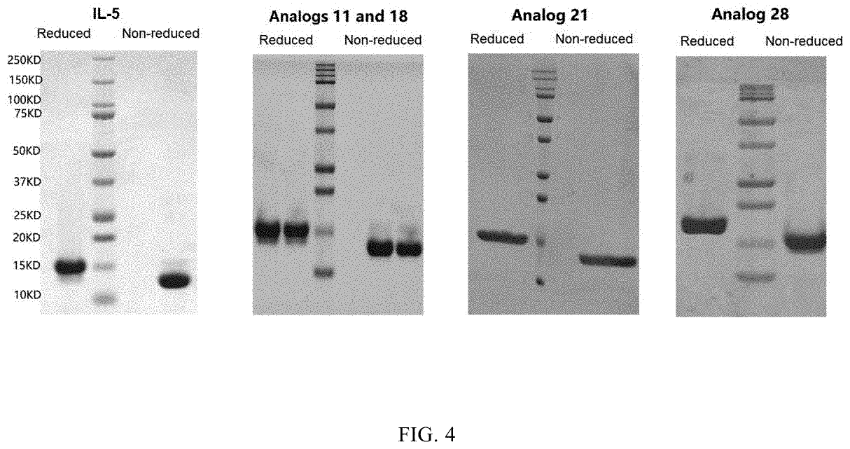

The IL-15 analogs 11, 18, 21 and 28 expressed by the inclusion bodies were dissolved in 8 M urea solution, and purified by ion exchange and reversed-phase chromatography (for details, see: Yunier Rodriguez-Alvarez et al, Preparative Biochemistry and Biotechnology, 47: 9, 889-900), to obtain relatively pure proteins. The SDS-PAGE electrophoretograms were shown in FIG. 4.

2.2 CTLL-2 Cell Proliferation Assay

The CTLL-2 cell proliferation assay is commonly used to detect the activity of immune cells stimulated by interleukin at the cellular level. Therefore, the biological activity of IL-15 analogs was determined herein by the proliferative effect of the wild-type IL-15 and IL-15 analogs on CTLL-2 cells.

1) Preparation of CTLL-2 Cells: The Cells were Resuspended in Media Containing FBS and Rat-T-Stim.

2) Loading: the cells were seeded in a 96-well culture plate at 0.1 mL per well. At the same time, the proteins samples of IL-15 analogs 11, 18, 21 and 28 to be tested (i.e., the proteins prepared in step 2.1) were diluted by multiples, respectively. 0.1 mL was added to each well, and 3 replicate wells were set for each dilution concentration. The control well for culture media was set (100 μL cells+100 μL culture media). The plate was incubated at 37° C. with 5% CO₂ for 72 hours.

3) MTS addition: 20 μL of CellTiter96® AQueous One Solution Reagent was added to each well, and the plate was incubated at 37° C. with 5% CO₂ for 2 to 4 hours.

4) Detection: the absorbance value (A) was measured at a wavelength of 490 nm with a microplate reader and the EC50 value was calculated.

The results were shown in Table 5. There was no significant difference in the cellular activity of the IL-15 analogs and wild-type IL-15.

TABLE 5
CTLL-2 cell viability assay

| | EC50 (ng/mL) |
|---|---|
| IL-15 | 0.05445 |
| Analog 11 | 0.05252 |
| Analog 18 | 0.05609 |
| Analog 21 | 0.05235 |
| Analog 28 | 0.05085 |

### Example 3: Preparation of Conjugates of IL-15 Analogs (Enzymatic Reaction Method)

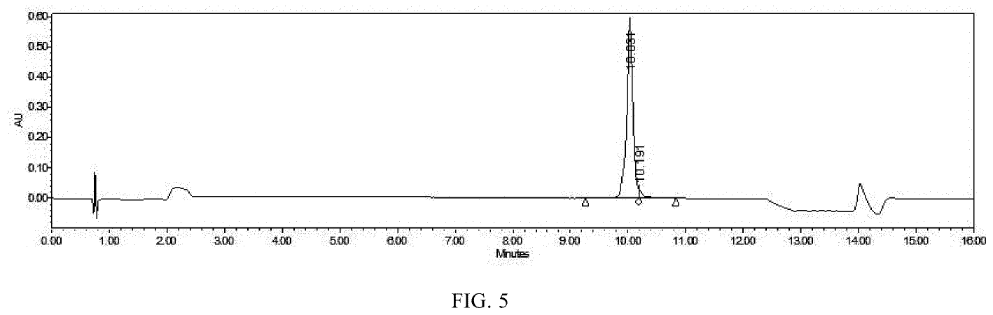

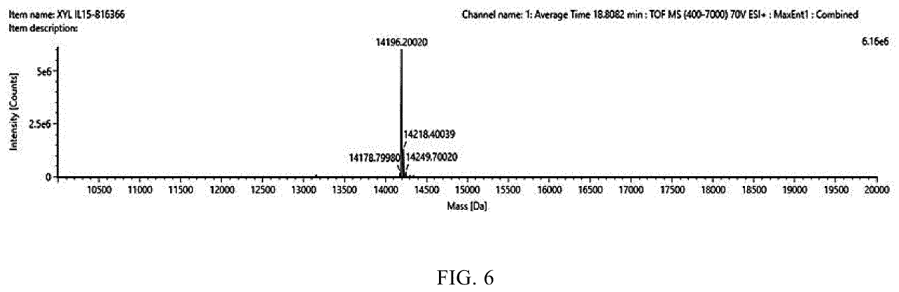

The purified IL-15 Analog 21 (IL-15-GS-LPETG-GSGGSHHHHHH) was used in this example. The fatty acid chain (816366) with GGG at the N-terminal was linked to IL-15-GS-LPETG-GSGGSHHHHHH by a ligation reaction catalyzed by the transpeptidase Sortase A. The reaction was carried out in a Sortase A: IL-15: fatty acid chain ratio of 1:6:30, wherein the reaction buffer was 50 mM Tris-HCl (1 mM CaCl, 150 mM NaCl, pH 8.0). After reacting at room temperature for 3 hours, purification was carried out. Reversed-phase chromatography C8 (Sepax Technologies, Inc.) was used for purification to separate the unconjugated IL-15 Analog 21 and the unreacted fatty acid chains from the conjugated products. The purity of the final product was determined by UPLC (FIG. 5) and LC-MS (FIG. 6). The results showed that IL-15 Analog 21 had been coupled to a fatty acid chain to form IL-15 Analog 21-816366.

### Example 4: Preparation of Conjugates of IL-15 Analog (Coupling the N-Terminal Amino Group of the IL-15 Analog to a Fatty Acid Chain)

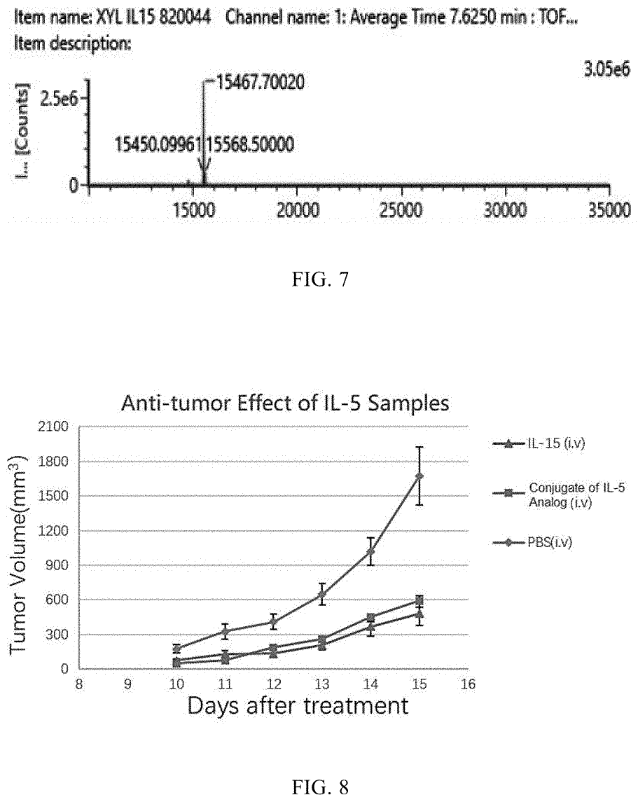

The purified IL-15 Analog 21 was coupled to a fatty acid chain with a succinimidyl ester (820044) through the amino group at N-terminal thereof at neutral pH. The reaction was carried out in an IL-15: fatty acid chain ratio of 1:1, wherein the reaction buffer was PBS at pH 7.2. After reacting at room temperature for 1 hour, purification was carried out. Reversed-phase chromatography C8 (Sepax Technologies, Inc.) was used for purification to separate the unconjugated IL-15 and the unreacted fatty acid chains from the conjugated products. The final product was identified by LC-MS. As shown in FIG. 7, IL-15 has been coupled to a fatty acid chain to form the IL-15 Analog 21-820044.

### Example 5: Binding Assay of the IL-15 Analog 21 and Conjugates of IL-15 Analog 21 to the IL15Rα Receptor

The conjugates of IL-15 analogs used in this example were prepared in Example 3.

In this example, biolayer interferomeory (BLI) was used to determine the affinity between the target protein and the receptor. For procedures, see Patricia Estep et al., High throughput solution Based measurement of antibody-antigen affinity and epitope binning, MAbs 2013, 5(2): 270-278. The receptor protein IL15Rα-His used in the experiment was produced by Leto Laboratories Co. Ltd. The formulation of buffer was: 10 mM HEPES, 150 mM NaCl, 3 mM EDTA, and 0.05% Tween 20. The receptor IL15Rα-His was pre-immobilized on a HISIK sensor (Pall Fortebio, Catalog #18-5120), followed by an established process comprising the steps of setting baseline, loading, baseline, association and dissociation. Data acquisition and analysis were carried out using the software Data acquisition 11.0 and Data analysis 11.0 installed with Octet RED96, respectively.

The results for affinity assay of the IL-15 analog and the conjugated products of IL-15 analogs and fatty acid chains to the IL15Rα receptors are shown in Table 6. As compared to the IL-15 analog before conjugation, the affinity of the conjugated products of IL-15 analog and fatty acid chains to the IL15Rα receptor did not change significantly.

TABLE 6
Results for affinity assay

| Name of Protein | Affinity to IL15Rα (M) |
|---|---|
| IL-15Analog 21 | 1.18E−09 |
| IL-15Analog 21-816366 | 6.6E−09 |
| IL-15Analog 21-823479 | 4E−09 |
| IL-15Analog 21-823480 | 8E−10 |

### Example 6: Half-Life of IL-15 Analog 21 and Conjugates of IL-15 Analog 21 in Mice

The conjugates of IL-15 analogs used in this example were prepared in Example 3.

Eight C57BL/6 mice were divided into 2 groups with 4 mice in each group. Blood was collected from two mice at each time point, and blood was collected cyclically. For one group, the mice were injected with IL-15 at 0.5 mg/kg via tail vein. Blood samples were collected immediately, and then at 10 min, 30 min, 1.5 h and 4 h after administration. For the other group, the mice were injected with long-acting IL-15 at 0.5 mg/kg via tail vein. Blood samples were collected immediately, and then at 1 h, 2 h and 4 h after administration. At each time point, 50 to 100 μL of blood was collected from eye orbit. Then serum was collected for ELISA assay of IL-15.

The calculation formula of half-life is: t_{1/2}=0.693/k, k=(lnc₀−lnc)/t. Upon calculation, the half-life of IL-15 was about 5 min, and the half-life of long-acting IL-15 could reach 1.5 h.

### Example 7: In Vivo Tumor Inhibition Assay for IL-15 Analog 21 and Conjugates of IL-15 Analog in Mice

The conjugates of IL-15 analogs used in this example were prepared in Example 3.

Fifteen C57BL/6 mice aged 6 to 8 weeks were randomly divided into 3 groups, namely the reagent control group (PBS), the IL-15 group and the long-acting IL-15 group, with 5 mice in each group. On Day 1, the mice were subcutaneously inoculated with B16-F10 cells on the back of the neck, with 2×10⁵/100 μL/mouse. On Days 4 to 8, the PBS group and the IL-15 group were administered intravenously (i.v.) for 5 consecutive days, respectively, at a dose of 20 μg/100 μL/mouse (IL-15 group) or 100 μL/mouse (PBS group). The IL-15 analog conjugate group was administered twice in the same way on Day 4 and Day 7, with the same dosage of 20 μg/100 μL/mouse each time. Tumor sizes were observed from Day 10 and continued for 6 days.

The results are shown in FIG. 8. As compared to the control group, both IL-15 and long-acting IL-15 exhibited significant tumor-inhibiting effects.

We claim:

1. An IL-15 analog, wherein an amino acid sequence of the IL-15 analog comprises an amino acid sequence of IL-15, and an amino acid sequence comprising at least one amino acid added to a C-terminal of the amino acid sequence of IL-15.

2. The IL-15 analog of claim 1, wherein the amino acid sequence added to the C-terminal of the amino acid sequence of IL-15 comprises at least one positively charged amino acid.

3. The IL-15 analog of claim 2, wherein the amino acid sequence of the IL-15 analog is characterized by:
IL-15-Xₐ-Y_{b}-Z_{c}
wherein X, Y and Z each represent an amino acid sequence added at the C-terminal of the amino acid sequence of IL-15, and a, b and c each represent a number of the amino acids;
wherein X and Z each are any amino acid or a combination of any amino acids, and a and c each are 0 to 20; and
wherein Y is the positively charged amino acid or a combination of any positively charged amino acids, or a combination of the positively charged amino acid and any other amino acids, and b is 1 to 7.

4. The IL-15 analog of claim 3, wherein the positively charged amino acid is H, R or K.

5. The IL-15 analog of claim 4, wherein X comprises at least one amino acid selected from the group consisting of V, I, P, L, E, A, S, C, T, and G.

6. The IL-15 analog of claim 3, wherein the amino acid sequence of the IL-15 analog is further characterized by:
IL-15-linker-Xₐ-Y_{b}-Z_{c}
wherein the linker represents a linker sequence between the amino acid sequence of IL-15 and the amino acid sequence added to the C-terminal thereof.

7. The IL-15 analog of claim 6, wherein the linker is (GGGGS)ₙ, (GS)ₙ or (GAPQ)ₙ, with n being 0 to 10.

8. The IL-15 analog of claim 7, wherein Xₐ comprises LPBTG with B being any amino acid, and the linker is (GS)ₙ.

9. The IL-15 analog of claim 1, wherein the amino acid sequence added to the C-terminal of the amino acid sequence of IL-15 is selected from the group consisting of: SEQ ID NOS: 122-150, KKK, KKC, K, and KK.

10. The IL-15 analog of claim 1, wherein the amino acid sequence of IL-15 is selected from the group consisting of:
1) the amino acid sequence shown in SEQ ID NO. 1;
2) an amino acid sequence derived from the amino acid sequence shown in SEQ ID NO. 1 through substitution, deletion, or addition of one or more amino acids; and
3) an amino acid being at least 80%, 85%, 90%, 91%, 92%, 93%, 94%, 95%, 96%, 97%, 98% or 99% identical to the amino acid sequence shown in SEQ ID NO. 1.

11. A nucleotide sequence encoding the IL-15 analog according to claim 1.

12. A method for preparing the IL-15 analog of claim 1, comprising expressing the IL-15 analog in a prokaryotic system.

13. The method of claim 12, comprising linking a nucleotide sequence encoding the IL-15 analog to a prokaryotic expression vector, and transferring a resulting vector into a prokaryotic expression host bacterium, and performing an induction to express the IL-15 analog.

14. A recombinant expression vector, comprising a nucleotide sequence encoding the IL-15 analog of claim 1.

15. A host bacterium transformed with a nucleotide sequence encoding the IL-15 analog of claim 1.

16. A conjugate of an IL-15 analog, comprising an amino acid sequence of the IL-15 analog linked with a fatty acid chain, wherein the amino acid sequence of the IL-15 analog comprises an amino acid sequence of IL-15, and an amino acid sequence comprising at least one amino acid added to a C-terminal of the amino acid sequence of IL-15.

17. The conjugate of the IL-15 analog of claim 16, wherein the amino acid sequence of the IL-15 analog is characterized by either one of:
1) IL-15-Xₐ-Y_{b}-Z_{c}
wherein X, Y and Z each represent an amino acid sequence added at the C-terminal of the amino acid sequence of IL-15, and a, b and c each represent a number of the amino acids; and
wherein X and Z each are any amino acid or a combination of any amino acids, and a and c each are 0 to 20; and
wherein Y is a positively charged amino acid or a combination of any positively charged amino acids, or a combination of a positively charged amino acid and any other amino acids, and b is 1 to 7; and
2) IL-15-linker-Xₐ-Y_{b}-Z_{c}
wherein the linker represents a linker sequence between the amino acid sequence of IL-15 and the amino acid sequence added to the C-terminal thereof.

18. The conjugate of the IL-15 analog of claim 17, wherein Xₐ comprises LPBTG with B being any amino acid, and the linker is (GS)ₙ with n being 0 to 10.

19. The conjugate of the IL-15 analog of claim 17, wherein the fatty acid chain is —(CH₂)ₘ—COOH, wherein m is 12 to 19.

20. The conjugate of the IL-15 analog of claim 17, wherein the IL-15 analog and the fatty acid chain are linked by in vitro coupling.

21. The conjugate of the IL-15 analog of claim 20, wherein the in vitro coupling is performed with one of the following methods:
1) coupling through an enzymatic reaction, wherein the fatty acid chain in the enzymatic reaction has GGG at an N-terminal;
2) coupling with free cysteine residues introduced into the amino acid sequence of the IL-15 analog; and
3) coupling with an amino group at an N-terminal of the amino acid sequence of the IL-15 analog.

22. The conjugate of the IL-15 analog of claim 21, wherein
when coupling through the enzymatic reaction, the fatty acid chain has 3 glycine residues at a terminal of the fatty acid chain;
when coupling with the free cysteine residues introduced into the amino acid sequence of the IL-15 analog, the fatty acid chain has a maleimide ester or a halogenated reactive group; and
when coupling with the amino group at the N-terminal of the amino acid sequence of the IL-15 analog, the fatty acid chain has an aldehyde group or a succinimidyl ester functional group.

23. The conjugate of the IL-15 analog of claim 16, wherein the IL-15 analog is obtained by adding a sequence comprising -GS-LPETG, as set forth in SEQ ID NO: 152, to the C-terminal of the amino acid sequence of IL-15.

24. The conjugate of the IL-15 analog of claim 16, wherein the fatty acid chain is selected from the group consisting of:
GGG-PEG2-Lys-(CH₂)₁₆-COOH,
NHS-PEG2-PEG2-γ-Glu-(CH₂)₁₇-COOH,
GGG-PEG4-PEG4-PEG4-Lys-(CH₂)₁₇-COOH,
GGG-PEG4-γ-Glu-γ-Glu-Lys-(CH₂)₁₇-COOH,
HOOC-(CH₂)₁₆-γ-Glu-γ-Glu-Lys-GGG,
HOOC-(CH₂)₁₆-CONH-γ-Glu-γ-Glu-PEG2-Lys-Br,
GGG-γ-Glu-C2DA-2OEG-γ-Glu-(CH₂)₁₇-COOH,
GGG-γ-Glu-C2DA-2OEG-γ-Glu-(CH₂)₁₉-COOH,
GGG-OEG-C2DA-2OEG-γ-Glu-(CH₂)₁₉-COOH,
GGG-OEG-C2DA-2OEG-γ-Glu-Trx-(CH₂)₁₉-COOH,
CHO-PEG2-PEG2-γ-Glu-(CH₂)₁₇-COOH,
and
Mal-C2DA-2OEG-γ-Glu-Tn-(CH₂)₁₉-COOH.


SEQUENCE LISTING (220 sequences)

SEQ ID NO: 1 - protein, 115 aa, Artificial Sequence
  Sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 345 bp, Artificial Sequence
  Sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagc

SEQ ID NO: 3 - protein, 133 aa, Artificial Sequence
  Sequence is synthesized
MNWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSGSLPETGGSGGSHHHHHH

SEQ ID NO: 4 - protein, 134 aa, Artificial Sequence
  Sequence is synthesized
MANWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSGSLPETGGSGGSHHHHHH

SEQ ID NO: 5 - protein, 133 aa, Artificial Sequence
  Sequence is synthesized
MNWVNVISDLKKIEDLIQSMHIRGDLYTESDVHPSCKVTAMKCFLLELQVISLESGDASIHDTVENLIILANNSLSSNANVTESGCKECEELEEKNIKEFLQSFVHIVQMFINTSGSLPETGGSGGSHHHHHH

SEQ ID NO: 6 - protein, 134 aa, Artificial Sequence
  Sequence is synthesized
MSNWVNVISDLKKIEDLIQSVHIRGDLYTESDVHPSCKVTAMKCFLLELQVISLESGDGSIHDTVENLIILAQQSLSSNANVTESGCKECEELEEKNIKEFLQSFVHIVQMFINTSGSLPETGGSGGSHHHHHH

SEQ ID NO: 7 - protein, 134 aa, Artificial Sequence
  Sequence is synthesized
MSNWVNVISDLKKIEDLIQSVHIRGDLYTESDVHPSCKVTAMKCFLLELQVISLESGDGSIHDTVENLIILAQQSLSSNANVTESGCKECEELSEKNIKEFLQSFVHIVQVFINTSGSLPETGGSGGSHHHHHH

SEQ ID NO: 8 - protein, 134 aa, Artificial Sequence
  Sequence is synthesized
MSNWVNVISDLRKIRGDIQSVHIDATLYTESDVHPSCRVTAMKCFLLELQVISLESGDASIHDTVENLIILANQSLSSNANVTESGCKECEELEEKNIKEFLQSFVHIVQLFIQTSGSLPETGGSGGSHHHHHH

SEQ ID NO: 9 - protein, 134 aa, Artificial Sequence
  Sequence is synthesized
MSNWVNVISDLRKIRGDLNAVHVDATLYTESDVHPSCRVTAMKCFLLELQVISLESGDASIHDTVENLIILANQSLSSNANVTESGCKECEELEEKNIKEFLQSFVHIVQLFIQTSGSLPETGGSGGSHHHHHH

SEQ ID NO: 10 - protein, 134 aa, Artificial Sequence
  Sequence is synthesized
MSNWVNVISDLRKIRGDIQSVHIDATLYTESDVHPSCRVTAMKCFLLELQVISLESGDASIHDTVENLIILANQSLAAQAQLTESGCKECEELEEKNIKEFLQSFVHIVQLFIQTSGSLPETGGSGGSHHHHHH

SEQ ID NO: 11 - protein, 134 aa, Artificial Sequence
  Sequence is synthesized
MSNWVNVISDLRKIRGDLNAVHVDATLYTESDVHPSCRVTAMKCFLLELQVISLESGDASIHDTVENLIILANQSLAAQAQLTESGCKECEELEEKNIKEFLQSFVHIVQLFIQTSGSLPETGGSGGSHHHHHH

SEQ ID NO: 12 - protein, 134 aa, Artificial Sequence
  Sequence is synthesized
MSNWVNVISDLRKIRGDIQSVHIDATLYTESDVHPSCRVTAMKCFLLELQLISLDSGDASIHETVEQLILLANQSLAAQAQLTESGCKECEELEEKNIKEFLQSFVHIVQLFIQTSGSLPETGGSGGSHHHHHH

SEQ ID NO: 13 - DNA, 396 bp, Artificial Sequence
  Sequence is synthesized
atgtgggtgaacgttatcagcgacctgaagaaaatcgaggatctgattcagagcatgcacattcgtggtgacctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacgcgaacgtgaccgagagcggctgcaaggaatgcgaggaactggaggaaaagaacatcaaagaattcctgcagagctttgtgcacatcgttcaaatgtttattaacaccagcggttctctgccggaaaccggtggttctggtggttctcaccaccaccaccaccac

SEQ ID NO: 14 - DNA, 402 bp, Artificial Sequence
  Sequence is synthesized
atgagcaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacgcgaacgtgaccgagagcggctgcaaggaatgcgaggaactggaggaaaagaacatcaaagaattcctgcagagctttgtgcacatcgttcaaatgtttattaacaccagcggttctctgccggaaaccggtggttctggtggttctcaccaccaccaccaccac

SEQ ID NO: 15 - DNA, 402 bp, Artificial Sequence
  Sequence is synthesized
atggccaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacgcgaacgtgaccgagagcggctgcaaggaatgcgaggaactggaggaaaagaacatcaaagaattcctgcagagctttgtgcacatcgttcaaatgtttattaacaccagcggttctctgccggaaaccggtggttctggtggttctcaccaccaccaccaccac

SEQ ID NO: 16 - DNA, 402 bp, Artificial Sequence
  Sequence is synthesized
atgagcaactgggtgaatgtgattagtgatctgaaaaaaattgaagatctgattcagagtgtgcatattagaggtgatctgtatactgaaagtgatgtgcatccaagctgcaaagtgactgccatgaaatgctttctgctggaactgcaagtgattagcctggaaagtggtgatggcagcattcatgatactgtggaaaacctgattattctggcacagcagagcctgagcagcaatgccaatgtgactgaaagtggctgcaaagaatgtgaagaactggaagaaaaaaacattaaagaatttctgcagagctttgtgcatattgtgcaaatgtttattaacactagtggcagcctgcctgaaactggtggcagtggtggcagccatcatcatcatcatcat

SEQ ID NO: 17 - DNA, 402 bp, Artificial Sequence
  Sequence is synthesized
atgagcaactgggtgaatgtgattagtgatctgaaaaaaattgaagatctgattcagagtgtgcatattagaggtgatctgtatactgaaagtgatgtgcatccaagctgcaaagtgactgccatgaaatgctttctgctggaactgcaagtgattagcctggaaagtggtgatggcagcattcatgatactgtggaaaacctgattattctggcacagcagagcctgagcagcaatgccaatgtgactgaaagtggctgcaaagaatgtgaagaactgagtgaaaaaaacattaaagaatttctgcagagctttgtgcatattgtgcaagtgtttattaacactagtggcagcctgcctgaaactggtggcagtggtggcagccatcatcatcatcatcat

SEQ ID NO: 18 - DNA, 402 bp, Artificial Sequence
  Sequence is synthesized
atgagcaactgggtgaatgtgattagtgatctgagaaaaattagaggtgatattcagagtgtgcatattgatgccaccctgtatactgaaagtgatgtgcatccaagctgcagagtgactgccatgaaatgctttctgctggaactgcaagtgattagcctggaaagtggtgatgcaagcattcatgatactgtggaaaacctgattattctggccaatcagagcctgagcagcaatgccaatgtgactgaaagtggctgcaaagaatgtgaagaactggaagaaaaaaacattaaagaatttctgcagagctttgtgcatattgtgcagctgtttattcagactagtggcagcctgcctgaaactggtggcagtggtggcagccatcatcatcatcatcat

SEQ ID NO: 19 - DNA, 402 bp, Artificial Sequence
  Sequence is synthesized
atgagcaactgggtgaatgtgattagtgatctgagaaaaattagaggtgatctgaatgcagtgcatgtggatgccaccctgtatactgaaagtgatgtgcatccaagctgcagagtgactgccatgaaatgctttctgctggaactgcaagtgattagcctggaaagtggtgatgcaagcattcatgatactgtggaaaacctgattattctggccaatcagagcctgagcagcaatgccaatgtgactgaaagtggctgcaaagaatgtgaagaactggaagaaaaaaacattaaagaatttctgcagagctttgtgcatattgtgcagctgtttattcagactagtggcagcctgcctgaaactggtggcagtggtggcagccatcatcatcatcatcat

SEQ ID NO: 20 - DNA, 402 bp, Artificial Sequence
  Sequence is synthesized
atgagcaactgggtgaatgtgattagtgatctgagaaaaattagaggtgatattcagagtgtgcatattgatgccaccctgtatactgaaagtgatgtgcatccaagctgcagagtgactgccatgaaatgctttctgctggaactgcaagtgattagcctggaaagtggtgatgcaagcattcatgatactgtggaaaacctgattattctggccaatcagagcctggcagcccaagcacagctgactgaaagtggctgcaaagaatgtgaagaactggaagaaaaaaacattaaagaatttctgcagagctttgtgcatattgtgcagctgtttattcagactagtggcagcctgcctgaaactggtggcagtggtggcagccatcatcatcatcatcat

SEQ ID NO: 21 - DNA, 402 bp, Artificial Sequence
  Sequence is synthesized
atgagcaactgggtgaatgtgattagtgatctgagaaaaattagaggtgatctgaatgcagtgcatgtggatgccaccctgtatactgaaagtgatgtgcatccaagctgcagagtgactgccatgaaatgctttctgctggaactgcaagtgattagcctggaaagtggtgatgcaagcattcatgatactgtggaaaacctgattattctggccaatcagagcctggcagcccaagcacagctgactgaaagtggctgcaaagaatgtgaagaactggaagaaaaaaacattaaagaatttctgcagagctttgtgcatattgtgcagctgtttattcagactagtggcagcctgcctgaaactggtggcagtggtggcagccatcatcatcatcatcat

SEQ ID NO: 22 - DNA, 402 bp, Artificial Sequence
  Sequence is synthesized
atgagcaactgggtgaatgtgattagtgatctgagaaaaattagaggtgatattcagagtgtgcatattgatgccaccctgtatactgaaagtgatgtgcatccaagctgcagagtgactgccatgaaatgctttctgctggaactgcagctgattagcctggatagtggtgatgcaagcattcatgaaactgtggaacagctgattctgctggccaatcagagcctggcagcccaagcacagctgactgaaagtggctgcaaagaatgtgaagaactggaagaaaaaaacattaaagaatttctgcagagctttgtgcatattgtgcagctgtttattcagactagtggcagcctgcctgaaactggtggcagtggtggcagccatcatcatcatcatcat

SEQ ID NO: 23 - DNA, 43 bp, Artificial Sequence
  Sequence is synthesized
taagaaggagatatacatatgaactgggtgaacgttatcagcg

SEQ ID NO: 24 - DNA, 58 bp, Artificial Sequence
  Sequence is synthesized
gtggtggtggtggtgagaaccagaaccagaaccgctggtgttaataaacatttgaacg

SEQ ID NO: 25 - DNA, 48 bp, Artificial Sequence
  Sequence is synthesized
gtggtggtggtggtgctcgagttagtggtggtggtggtggtgagaacc

SEQ ID NO: 26 - DNA, 43 bp, Artificial Sequence; identical to SEQ ID NO: 23
  Sequence is synthesized

SEQ ID NO: 27 - DNA, 49 bp, Artificial Sequence
  Sequence is synthesized
gtggtggtggtggtggtgagaaccgctggtgttaataaacatttgaacg

SEQ ID NO: 28 - DNA, 44 bp, Artificial Sequence
  Sequence is synthesized
gtggtggtggtggtgctcgagttagtggtggtggtggtggtgag

SEQ ID NO: 29 - DNA, 43 bp, Artificial Sequence; identical to SEQ ID NO: 23
  Sequence is synthesized

SEQ ID NO: 30 - DNA, 49 bp, Artificial Sequence
  Sequence is synthesized
acgctttttggtgctagcaagtgggctggtgttaataaacatttgaacg

SEQ ID NO: 31 - DNA, 45 bp, Artificial Sequence
  Sequence is synthesized
gtggtggtggtggtgctcgagttaacgctttttggtgctagcaag

SEQ ID NO: 32 - DNA, 43 bp, Artificial Sequence; identical to SEQ ID NO: 23
  Sequence is synthesized

SEQ ID NO: 33 - DNA, 49 bp, Artificial Sequence
  Sequence is synthesized
cttctttttagcagactttggaaggctggtgttaataaacatttgaacg

SEQ ID NO: 34 - DNA, 49 bp, Artificial Sequence
  Sequence is synthesized
gtggtggtggtggtgctcgagttacttctttttagcagactttggaagg

SEQ ID NO: 35 - DNA, 43 bp, Artificial Sequence; identical to SEQ ID NO: 23
  Sequence is synthesized

SEQ ID NO: 36 - DNA, 46 bp, Artificial Sequence
  Sequence is synthesized
cttcttttttttcttctttttgctggtgttaataaacatttgaacg

SEQ ID NO: 37 - DNA, 50 bp, Artificial Sequence
  Sequence is synthesized
gtggtggtggtggtgctcgagttacttcttttttttcttctttttgctgg

SEQ ID NO: 38 - DNA, 43 bp, Artificial Sequence; identical to SEQ ID NO: 23
  Sequence is synthesized

SEQ ID NO: 39 - DNA, 59 bp, Artificial Sequence
  Sequence is synthesized
attccacaagctgtggagcaccctgtggagcaccgctggtgttaataaacatttgaacg

SEQ ID NO: 40 - DNA, 59 bp, Artificial Sequence
  Sequence is synthesized
gtggtggtggtggtgctcgagttaatgatggtgtgcagattccacaagctgtggagcac

SEQ ID NO: 41 - DNA, 43 bp, Artificial Sequence; identical to SEQ ID NO: 23
  Sequence is synthesized

SEQ ID NO: 42 - DNA, 55 bp, Artificial Sequence
  Sequence is synthesized
tttgtggtgagcgctagatacaaggctaccgctggtgttaataaacatttgaacg

SEQ ID NO: 43 - DNA, 47 bp, Artificial Sequence
  Sequence is synthesized
gtggtggtggtggtgctcgagttatttgtggtgagcgctagatacaa

SEQ ID NO: 44 - DNA, 43 bp, Artificial Sequence; identical to SEQ ID NO: 23
  Sequence is synthesized

SEQ ID NO: 45 - DNA, 55 bp, Artificial Sequence
  Sequence is synthesized
tttgcgacggtggtgttcgataaggctaccgctggtgttaataaacatttgaacg

SEQ ID NO: 46 - DNA, 43 bp, Artificial Sequence
  Sequence is synthesized
gtggtggtggtggtgctcgagttatttgcgacggtggtgttcg

SEQ ID NO: 47 - DNA, 43 bp, Artificial Sequence; identical to SEQ ID NO: 23
  Sequence is synthesized

SEQ ID NO: 48 - DNA, 55 bp, Artificial Sequence
  Sequence is synthesized
ctttttcttacggtgttctacaatgctaccgctggtgttaataaacatttgaacg

SEQ ID NO: 49 - DNA, 50 bp, Artificial Sequence
  Sequence is synthesized
gtggtggtggtggtgctcgagttactttttcttacggtgttctacaatgc

SEQ ID NO: 50 - DNA, 43 bp, Artificial Sequence; identical to SEQ ID NO: 23
  Sequence is synthesized

SEQ ID NO: 51 - DNA, 55 bp, Artificial Sequence
  Sequence is synthesized
acggcgacgaccagtttttggtacgctaccgctggtgttaataaacatttgaacg

SEQ ID NO: 52 - DNA, 43 bp, Artificial Sequence
  Sequence is synthesized
gtggtggtggtggtgctcgagttaacggcgacgaccagttttt

SEQ ID NO: 53 - DNA, 43 bp, Artificial Sequence; identical to SEQ ID NO: 23
  Sequence is synthesized

SEQ ID NO: 54 - DNA, 52 bp, Artificial Sequence
  Sequence is synthesized
ctttttaccgctagcaaccaggctaccgctggtgttaataaacatttgaacg

SEQ ID NO: 55 - DNA, 46 bp, Artificial Sequence
  Sequence is synthesized
gtggtggtggtggtgctcgagttactttttaccgctagcaaccagg

SEQ ID NO: 56 - DNA, 43 bp, Artificial Sequence; identical to SEQ ID NO: 23
  Sequence is synthesized

SEQ ID NO: 57 - DNA, 55 bp, Artificial Sequence
  Sequence is synthesized
atgatggtgaccagatttacgatggctaccgctggtgttaataaacatttgaacg

SEQ ID NO: 58 - DNA, 48 bp, Artificial Sequence
  Sequence is synthesized
gtggtggtggtggtgctcgagttaatgatggtgaccagatttacgatg

SEQ ID NO: 59 - DNA, 43 bp, Artificial Sequence; identical to SEQ ID NO: 23
  Sequence is synthesized

SEQ ID NO: 60 - DNA, 55 bp, Artificial Sequence
  Sequence is synthesized
cttgtgacgaccagtttttggaaggctaccgctggtgttaataaacatttgaacg

SEQ ID NO: 61 - DNA, 46 bp, Artificial Sequence
  Sequence is synthesized
gtggtggtggtggtgctcgagttacttgtgacgaccagtttttgga

SEQ ID NO: 62 - DNA, 43 bp, Artificial Sequence; identical to SEQ ID NO: 23
  Sequence is synthesized

SEQ ID NO: 63 - DNA, 49 bp, Artificial Sequence
  Sequence is synthesized
atggcgacgaccagtcttctttttgctggtgttaataaacatttgaacg

SEQ ID NO: 64 - DNA, 45 bp, Artificial Sequence
  Sequence is synthesized
gtggtggtggtggtgctcgagttaatggcgacgaccagtcttctt

SEQ ID NO: 65 - DNA, 43 bp, Artificial Sequence; identical to SEQ ID NO: 23
  Sequence is synthesized

SEQ ID NO: 66 - DNA, 49 bp, Artificial Sequence
  Sequence is synthesized
cttatgacgaccagaacgtggaaggctggtgttaataaacatttgaacg

SEQ ID NO: 67 - DNA, 46 bp, Artificial Sequence
  Sequence is synthesized
gtggtggtggtggtgctcgagttacttatgacgaccagaacgtgga

SEQ ID NO: 68 - DNA, 43 bp, Artificial Sequence; identical to SEQ ID NO: 23
  Sequence is synthesized

SEQ ID NO: 69 - DNA, 49 bp, Artificial Sequence
  Sequence is synthesized
gtgatgatggtgagtttcaaccaggctggtgttaataaacatttgaacg

SEQ ID NO: 70 - DNA, 48 bp, Artificial Sequence
  Sequence is synthesized
gtggtggtggtggtgctcgagttagtgatgatggtgagtttcaaccag

SEQ ID NO: 71 - DNA, 43 bp, Artificial Sequence; identical to SEQ ID NO: 23
  Sequence is synthesized

SEQ ID NO: 72 - DNA, 49 bp, Artificial Sequence
  Sequence is synthesized
atgatggtgagtttctggacgaacgctggtgttaataaacatttgaacg

SEQ ID NO: 73 - DNA, 47 bp, Artificial Sequence
  Sequence is synthesized
gtggtggtggtggtgctcgagttaatgatggtgagtttctggacgaa

SEQ ID NO: 74 - DNA, 43 bp, Artificial Sequence; identical to SEQ ID NO: 23
  Sequence is synthesized

SEQ ID NO: 75 - DNA, 34 bp, Artificial Sequence
  Sequence is synthesized
cttttttttgctggtgttaataaacatttgaacg

SEQ ID NO: 76 - DNA, 50 bp, Artificial Sequence
  Sequence is synthesized
gtggtggtggtggtgctcgagttacttttttttgctggtgttaataaaca

SEQ ID NO: 77 - DNA, 43 bp, Artificial Sequence; identical to SEQ ID NO: 23
  Sequence is synthesized

SEQ ID NO: 78 - DNA, 40 bp, Artificial Sequence
  Sequence is synthesized
atgatgatggtgacggctggtgttaataaacatttgaacg

SEQ ID NO: 79 - DNA, 44 bp, Artificial Sequence
  Sequence is synthesized
gtggtggtggtggtgctcgagttaatgatgatggtgacggctgg

SEQ ID NO: 80 - DNA, 43 bp, Artificial Sequence; identical to SEQ ID NO: 23
  Sequence is synthesized

SEQ ID NO: 81 - DNA, 49 bp, Artificial Sequence
  Sequence is synthesized
atgatgatggtgagtttcacgcttgctggtgttaataaacatttgaacg

SEQ ID NO: 82 - DNA, 47 bp, Artificial Sequence
  Sequence is synthesized
gtggtggtggtggtgctcgagttaatgatgatggtgagtttcacgct

SEQ ID NO: 83 - DNA, 43 bp, Artificial Sequence; identical to SEQ ID NO: 23
  Sequence is synthesized

SEQ ID NO: 84 - DNA, 52 bp, Artificial Sequence
  Sequence is synthesized
accaccagaaccaccggtttccggcagagaaccgctggtgttaataaacatt

SEQ ID NO: 85 - DNA, 57 bp, Artificial Sequence
  Sequence is synthesized
gtggtggtggtggtgctcgagttagtggtggtggtggtggtgagaaccaccagaacc

SEQ ID NO: 86 - DNA, 43 bp, Artificial Sequence; identical to SEQ ID NO: 23
  Sequence is synthesized

SEQ ID NO: 87 - DNA, 49 bp, Artificial Sequence
  Sequence is synthesized
cttctttttaccagtttcaaggtggctggtgttaataaacatttgaacg

SEQ ID NO: 88 - DNA, 49 bp, Artificial Sequence
  Sequence is synthesized
gtggtggtggtggtgctcgagttacttctttttaccagtttcaaggtgg

SEQ ID NO: 89 - DNA, 43 bp, Artificial Sequence; identical to SEQ ID NO: 23
  Sequence is synthesized

SEQ ID NO: 90 - DNA, 49 bp, Artificial Sequence
  Sequence is synthesized
acggcgacgaccagattcaacatggctggtgttaataaacatttgaacg

SEQ ID NO: 91 - DNA, 43 bp, Artificial Sequence
  Sequence is synthesized
gtggtggtggtggtgctcgagttaacggcgacgaccagattca

SEQ ID NO: 92 - DNA, 43 bp, Artificial Sequence; identical to SEQ ID NO: 23
  Sequence is synthesized

SEQ ID NO: 93 - DNA, 49 bp, Artificial Sequence
  Sequence is synthesized
cttctttttaccagtatgacgacggctggtgttaataaacatttgaacg

SEQ ID NO: 94 - DNA, 49 bp, Artificial Sequence
  Sequence is synthesized
gtggtggtggtggtgctcgagttacttctttttaccagtatgacgacgg

SEQ ID NO: 95 - DNA, 43 bp, Artificial Sequence; identical to SEQ ID NO: 23
  Sequence is synthesized

SEQ ID NO: 96 - DNA, 49 bp, Artificial Sequence
  Sequence is synthesized
atgatggtgaccagtcttaacatggctggtgttaataaacatttgaacg

SEQ ID NO: 97 - DNA, 49 bp, Artificial Sequence
  Sequence is synthesized
gtggtggtggtggtgctcgagttaatgatggtgaccagtcttaacatgg

SEQ ID NO: 98 - DNA, 43 bp, Artificial Sequence; identical to SEQ ID NO: 23
  Sequence is synthesized

SEQ ID NO: 99 - DNA, 49 bp, Artificial Sequence
  Sequence is synthesized
atgatgacgaccagacttaacatggctggtgttaataaacatttgaacg

SEQ ID NO: 100 - DNA, 49 bp, Artificial Sequence
  Sequence is synthesized
gtggtggtggtggtgctcgagttaatgatgacgaccagacttaacatgg

SEQ ID NO: 101 - DNA, 43 bp, Artificial Sequence; identical to SEQ ID NO: 23
  Sequence is synthesized

SEQ ID NO: 102 - DNA, 49 bp, Artificial Sequence
  Sequence is synthesized
gtgacgatggctagacttaacatggctggtgttaataaacatttgaacg

SEQ ID NO: 103 - DNA, 49 bp, Artificial Sequence
  Sequence is synthesized
gtggtggtggtggtgctcgagttagtgacgatggctagacttaacatgg

SEQ ID NO: 104 - DNA, 43 bp, Artificial Sequence; identical to SEQ ID NO: 23
  Sequence is synthesized

SEQ ID NO: 105 - DNA, 55 bp, Artificial Sequence
  Sequence is synthesized
gctaccgctaccgctaccgctaccgctaccgctggtgttaataaacatttgaacg

SEQ ID NO: 106 - DNA, 43 bp, Artificial Sequence
  Sequence is synthesized
atgatggtgaccagactttaccaggctaccgctaccgctaccg

SEQ ID NO: 107 - DNA, 48 bp, Artificial Sequence
  Sequence is synthesized
gtggtggtggtggtgctcgagttaatgatggtgaccagactttaccag

SEQ ID NO: 108 - DNA, 43 bp, Artificial Sequence; identical to SEQ ID NO: 23
  Sequence is synthesized

SEQ ID NO: 109 - DNA, 49 bp, Artificial Sequence
  Sequence is synthesized
cttatggtgaccgctctttggacggctggtgttaataaacatttgaacg

SEQ ID NO: 110 - DNA, 46 bp, Artificial Sequence
  Sequence is synthesized
gtggtggtggtggtgctcgagttacttatggtgaccgctctttgga

SEQ ID NO: 111 - DNA, 43 bp, Artificial Sequence; identical to SEQ ID NO: 23
  Sequence is synthesized

SEQ ID NO: 112 - DNA, 58 bp, Artificial Sequence
  Sequence is synthesized
gtggtggtggtggtgctcgagttaacactttttgctggtgttaataaacatttgaacg

SEQ ID NO: 113 - DNA, 43 bp, Artificial Sequence; identical to SEQ ID NO: 23
  Sequence is synthesized

SEQ ID NO: 114 - DNA, 49 bp, Artificial Sequence
  Sequence is synthesized
atggtgtttaccagccttgtgaaggctggtgttaataaacatttgaacg

SEQ ID NO: 115 - DNA, 47 bp, Artificial Sequence
  Sequence is synthesized
gtggtggtggtggtgctcgagttaatggtgtttaccagccttgtgaa

SEQ ID NO: 116 - DNA, 43 bp, Artificial Sequence; identical to SEQ ID NO: 23
  Sequence is synthesized

SEQ ID NO: 117 - DNA, 52 bp, Artificial Sequence
  Sequence is synthesized
gtggtggtggtggtgctcgagttatttgctggtgttaataaacatttgaacg

SEQ ID NO: 118 - DNA, 43 bp, Artificial Sequence; identical to SEQ ID NO: 23
  Sequence is synthesized

SEQ ID NO: 119 - DNA, 55 bp, Artificial Sequence
  Sequence is synthesized
gtggtggtggtggtgctcgagttactttttgctggtgttaataaacatttgaacg

SEQ ID NO: 120 - protein, 5 aa, Artificial Sequence
  The sequence is synthesized
GGGGS

SEQ ID NO: 121 - protein, 4 aa, Artificial Sequence
  The sequence is synthesized
GAPQ

SEQ ID NO: 122 - protein, 12 aa, Artificial Sequence
  The sequence is synthesized
GSGSGSHHHHHH

SEQ ID NO: 123 - protein, 8 aa, Artificial Sequence
  The sequence is synthesized
GSHHHHHH

SEQ ID NO: 124 - protein, 8 aa, Artificial Sequence
  The sequence is synthesized
PLASTKKR

SEQ ID NO: 125 - protein, 8 aa, Artificial Sequence
  The sequence is synthesized
LPKSAKKK

SEQ ID NO: 126 - protein, 7 aa, Artificial Sequence
  The sequence is synthesized
KKKKKKK

SEQ ID NO: 127 - protein, 16 aa, Artificial Sequence
  The sequence is synthesized
GAPQGAPQLVESAHHH

SEQ ID NO: 128 - protein, 10 aa, Artificial Sequence
  The sequence is synthesized
GSLVSSAHHK

SEQ ID NO: 129 - protein, 10 aa, Artificial Sequence
  The sequence is synthesized
GSLIEHHRRK

SEQ ID NO: 130 - protein, 10 aa, Artificial Sequence
  The sequence is synthesized
GSIVEHRKKK

SEQ ID NO: 131 - protein, 10 aa, Artificial Sequence
  The sequence is synthesized
GSVPKTGRRR

SEQ ID NO: 132 - protein, 9 aa, Artificial Sequence
  The sequence is synthesized
GSLVASGKK

SEQ ID NO: 133 - protein, 10 aa, Artificial Sequence
  The sequence is synthesized
GSHRKSGHHH

SEQ ID NO: 134 - protein, 10 aa, Artificial Sequence
  The sequence is synthesized
GSLPKTGRHK

SEQ ID NO: 135 - protein, 8 aa, Artificial Sequence
  The sequence is synthesized
KKKTGRRH

SEQ ID NO: 136 - protein, 8 aa, Artificial Sequence
  The sequence is synthesized
LPRSGRHK

SEQ ID NO: 137 - protein, 8 aa, Artificial Sequence
  The sequence is synthesized
LVETHHHH

SEQ ID NO: 138 - protein, 8 aa, Artificial Sequence
  The sequence is synthesized
VRPETHHH

SEQ ID NO: 139 - protein, 5 aa, Artificial Sequence
  The sequence is synthesized
RHHHH

SEQ ID NO: 140 - protein, 8 aa, Artificial Sequence
  The sequence is synthesized
KRETHHHH

SEQ ID NO: 141 - protein, 18 aa, Artificial Sequence
  The sequence is synthesized
GSLPETGGSGGSHHHHHH

SEQ ID NO: 142 - protein, 8 aa, Artificial Sequence
  The sequence is synthesized
HLETGKKK

SEQ ID NO: 143 - protein, 8 aa, Artificial Sequence
  The sequence is synthesized
HVESGRRR

SEQ ID NO: 144 - protein, 8 aa, Artificial Sequence
  The sequence is synthesized
RRHTGKKK

SEQ ID NO: 145 - protein, 8 aa, Artificial Sequence
  The sequence is synthesized
HVKTGHHH

SEQ ID NO: 146 - protein, 8 aa, Artificial Sequence
  The sequence is synthesized
HVKTGHHH

SEQ ID NO: 147 - protein, 8 aa, Artificial Sequence
  The sequence is synthesized
HVKSSHRH

SEQ ID NO: 148 - protein, 18 aa, Artificial Sequence
  The sequence is synthesized
GSGSGSGSGSLVKSGHHH

SEQ ID NO: 149 - protein, 8 aa, Artificial Sequence
  The sequence is synthesized
RPKSGHHK

SEQ ID NO: 150 - protein, 8 aa, Artificial Sequence
  The sequence is synthesized
LHKAGKHH

SEQ ID NO: 151 - protein, 5 aa, Artificial Sequence
  The sequence is synthesized
LPBTG

SEQ ID NO: 152 - protein, 7 aa, Artificial Sequence
  The sequence is synthesized
GSLPETG

SEQ ID NO: 153 - protein, 5 aa, Artificial Sequence
  The sequence is synthesized
LPETG

SEQ ID NO: 154 - protein, 127 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSHHHHHH

SEQ ID NO: 155 - DNA, 381 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggttctggttctggttctcaccaccaccaccaccac

SEQ ID NO: 156 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSHHHHHH

SEQ ID NO: 157 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggttctcaccaccaccaccaccac

SEQ ID NO: 158 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSPLASTKKR

SEQ ID NO: 159 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcccacttgctagcaccaaaaagcgt

SEQ ID NO: 160 - protein, 115 aa, Artificial Sequence; identical to SEQ ID NO: 1
  The sequence is synthesized

SEQ ID NO: 161 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccttccaaagtctgctaaaaagaag

SEQ ID NO: 162 - protein, 122 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKKKKKKK

SEQ ID NO: 163 - DNA, 366 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcaaaaagaagaaaaaaaagaag

SEQ ID NO: 164 - protein, 131 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPQGAPQLVESAHHH

SEQ ID NO: 165 - DNA, 393 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtgctccacagggtgctccacagcttgtggaatctgcacaccatcat

SEQ ID NO: 166 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSLVSSAHHK

SEQ ID NO: 167 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagccttgtatctagcgctcaccacaaa

SEQ ID NO: 168 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSLIEHHRRK

SEQ ID NO: 169 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagccttatcgaacaccaccgtcgcaaa

SEQ ID NO: 170 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSIVEHRKKK

SEQ ID NO: 171 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagcattgtagaacaccgtaagaaaaag

SEQ ID NO: 172 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSVPKTGRRR

SEQ ID NO: 173 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagcgtaccaaaaactggtcgtcgccgt

SEQ ID NO: 174 - protein, 124 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSLVASGKK

SEQ ID NO: 175 - DNA, 372 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagcctggttgctagcggtaaaaag

SEQ ID NO: 176 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSHRKSGHHH

SEQ ID NO: 177 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagccatcgtaaatctggtcaccatcat

SEQ ID NO: 178 - protein, 125 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSLPKTGRHK

SEQ ID NO: 179 - DNA, 375 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagccttccaaaaactggtcgtcacaag

SEQ ID NO: 180 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKKKTGRRH

SEQ ID NO: 181 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcaaaaagaagactggtcgtcgccat

SEQ ID NO: 182 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLPRSGRHK

SEQ ID NO: 183 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccttccacgttctggtcgtcataag

SEQ ID NO: 184 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLVETHHHH

SEQ ID NO: 185 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcctggttgaaactcaccatcatcac

SEQ ID NO: 186 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSVRPETHHH

SEQ ID NO: 187 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcgttcgtccagaaactcaccatcat

SEQ ID NO: 188 - protein, 118 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKKK

SEQ ID NO: 189 - DNA, 354 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcaaaaaaaag

SEQ ID NO: 190 - protein, 120 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRHHHH

SEQ ID NO: 191 - DNA, 360 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccgtcaccatcatcat

SEQ ID NO: 192 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKRETHHHH

SEQ ID NO: 193 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcaagcgtgaaactcaccatcatcat

SEQ ID NO: 194 - protein, 133 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSLPETGGSGGSHHHHHH

SEQ ID NO: 195 - DNA, 399 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggttctctgccggaaaccggtggttctggtggttctcaccaccaccaccaccac

SEQ ID NO: 196 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHLETGKKK

SEQ ID NO: 197 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccaccttgaaactggtaaaaagaag

SEQ ID NO: 198 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHVESGRRR

SEQ ID NO: 199 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccatgttgaatctggtcgtcgccgt

SEQ ID NO: 200 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRRHTGKKK

SEQ ID NO: 201 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccgtcgtcatactggtaaaaagaag

SEQ ID NO: 202 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHVKTGHHH

SEQ ID NO: 203 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccatgttaagactggtcaccatcat

SEQ ID NO: 204 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHVKSGRHH

SEQ ID NO: 205 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccatgttaagtctggtcgtcatcat

SEQ ID NO: 206 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHVKSSHRH

SEQ ID NO: 207 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccatgttaagtctagccatcgtcac

SEQ ID NO: 208 - protein, 133 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSGSGSLVKSGHHH

SEQ ID NO: 209 - DNA, 399 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcggtagcggtagcggtagcggtagcggtagcctggtaaagtctggtcaccatcat

SEQ ID NO: 210 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRPKSGHHK

SEQ ID NO: 211 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccgtccaaagagcggtcaccataag

SEQ ID NO: 212 - protein, 118 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKKC

SEQ ID NO: 213 - DNA, 354 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagcaaaaagtgt

SEQ ID NO: 214 - protein, 123 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLHKAGKHH

SEQ ID NO: 215 - DNA, 369 bp, Artificial Sequence
  The sequence is synthesized
atgaactgggtgaacgttatcagcgacctgaagaaaatcgaggatctgattcagagcatgcacattgacgcgaccctgtacaccgaaagcgatgtgcacccgagctgcaaggttaccgcgatgaaatgcttcctgctggagctgcaagtgatcagcctggaaagcggtgacgcgagcattcacgataccgttgagaacctgatcattctggcgaacaacagcctgagcagcaacggtaacgtgaccgagagcggctgcaaggaatgcgaggaactggaggaaaagaacatcaaagaattcctgcagagctttgtgcacatcgttcaaatgtttattaacaccagccttcacaaggctggtaaacaccat

SEQ ID NO: 216 - protein, 115 aa, Artificial Sequence; identical to SEQ ID NO: 1
  The sequence is synthesized

SEQ ID NO: 217 - DNA, 345 bp, Artificial Sequence; identical to SEQ ID NO: 2
  The sequence is synthesized

SEQ ID NO: 218 - protein, 117 aa, Artificial Sequence
  The sequence is synthesized
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSKK

SEQ ID NO: 219 - DNA, 345 bp, Artificial Sequence; identical to SEQ ID NO: 2
  The sequence is synthesized

SEQ ID NO: 220 - protein, 19 aa, Artificial Sequence
  The sequence is synthesized
GSLPETGGGSGGSHHHHHH